(19) United States
(12) Patent Application Publication
(10) Pub. No.: US 2026/0335251 A1
(43) Pub. Date: Oct. 29, 2026

(54) RED BLOOD CELL-HITCHHIKING IONIC LIQUID COATED NANOPARTICLES FOR CROSSING THE BLOOD BRAIN BARRIER

(71) Applicant: University of Mississippi, University, MS (US)

(72) Inventors: Eden Elizabeth Louise TANNER, University, MS (US); Christine HAMADANI, University, MS (US); Jason PARIS, University, MS (US)

(21) Appl. No.: 19/106,758

(22) PCT Filed: Aug. 21, 2023
(86) PCT No.: PCT/US2023/072545

§ 371 Date: Feb. 26, 2025

Related U.S. Application Data
(60) Provisional application No. 63/373,609, filed on Aug. 26, 2022.

(51) Int. Cl. A61K 9/51; A61K 9/00; A61K 31/52

(52) U.S. Cl. CPC A61K 9/5153; A61K 9/0019; A61K 9/5146; A61K 31/52

(57) ABSTRACT

In one aspect, this disclosure relates to nanoparticle compositions comprising a core a plurality of nanoparticles, each nanoparticle of the plurality comprising a core, wherein the core comprises a biocompatible copolymer, and each nanoparticle of the plurality further comprising an ionic liquid coating surrounding the core; methods of making the same; pharmaceutical compositions comprising same; and methods of treating neurological diseases and disorders using same. In one aspect, the biocompatible copolymer can be poly(lactic-co-glycolic acid). In another aspect, the nanoparticle compositions can include one or more pharmaceutical agents including, but not limited to, antiretroviral agents. In still another aspect, the ionic liquids can include choline and an anion derived from a substituted or unsubstituted C2-C20 linear or branched fatty acid. In any of these aspects, the nanoparticle compositions are capable of crossing the blood brain barrier.

### CROSS-REFERENCE TO RELATED APPLICATIONS

This application claims the benefit of U.S. Provisional Application No. 63/373,609, filed on Aug. 26, 2022, which is incorporated herein by reference in its entirety.

### BACKGROUND

Drug delivery to the brain remains an unsolved challenge. For infectious diseases such as HIV, the brain can act as a latent reservoir, untouched by systemic administration of anti-retrovirals and leaving patients with worsening neurological deficits. The effectiveness of HIV therapy via antiretroviral therapeutics is limited given the poor accumulation of antiretroviral drugs in the central nervous system, which serves as a reservoir for active and latent viral infection. Nanoparticles (NPs) provide an innovative solution for enhancing drug targeting and delivery, however rapid clearance of nanoparticles from circulation post-intravenous (IV) injection hinders clinical utility: less than 1% of IV-injected NPs reach their destination in diseased tissue.

About 1.2 million people in the United States are currently living with human immunodeficiency virus (HIV). Among patients infected with HIV-1, combination antiretroviral therapy (cART) has greatly reduced acquired immunodeficiency syndrome (AIDS) risk. However, infected individuals contend with pervasive HIV-associated neurocognitive and neuropsychiatric disorders, including deficits in learning and memory, behavioral inhibition, and affective well-being (hereafter, collectively referred to as “neuroHIV”). cART cannot eradicate neuroHIV, given that it poorly accumulates in the central nervous system (CNS) and does not target latent CNS viral reservoirs (microglia/macrophages and astrocytes). As such, ~50% of HIV+ patients suffer from neuro-cognitive and -psychiatric disorders.

Current antiretroviral therapies cannot eradicate HIV. This is due in part to their incapacity to accumulate well in HIV-infected reservoirs within the body, including the central nervous system, and their incapacity to target latently-infected cells. Nanoparticles have been utilized in an attempt to increase the delivery of antiretrovirals to the central nervous system by ‘hitchhiking’ on red blood cells, however this technology is presently limited, requiring mechanical combination of nanoparticles and red blood cells outside of the body followed by intravenous (IV) infusion back to the host. Current delivery rates to the brain are limited up to a maximum of ~20% of the injected dose, which typically initiates clearance around 6 hours.

Ionic liquids (ILs) are composed of bulky, asymmetric anions and cations and are liquid at temperatures less than 100° C. When ILs are synthesized from bioinspired ingredients, they retain high biocompatibility and have been used in a variety of drug delivery applications, including transdermal, buccal, subcutaneous, and oral delivery of therapeutics. However, when injected into the tail vein, therapeutic agents including ILs accumulate primarily in the lungs and other organs. In order to effectively treat neuro-HIV and other diseases and disorders affecting the brain, therapeutic agents must be developed that are capable of being delivered across the blood brain barrier (BBB).

Disclosed herein are bioinspired ionic liquid-coated nanoparticles for in situ hitchhiking onto red blood cells, which enables ~50% delivery efficacy to the brain. The ionic liquid-coated nanoparticles show preferential accumulation in microglia over endothelial cells post-delivery. Also disclosed herein are ionic-liquid-coated nanoparticles loaded with abacavir, a leading antiretroviral agent, as well as other pharmaceutical agents; the abacavir (ABC) retains its antiviral potency in human peripheral blood mononuclear cells (PMBCs) and is non-toxic. Additionally, the IL-coated NP itself shows anti-viremic activity in the absence of abacavir.

### SUMMARY

In accordance with the purpose(s) of the present disclosure, as embodied and broadly described herein, the disclosure, in one aspect, relates to nanoparticle compositions comprising a core a plurality of nanoparticles, each nanoparticle of the plurality comprising a core, wherein the core comprises a biocompatible copolymer, and each nanoparticle of the plurality further comprising an ionic liquid coating surrounding the core; methods of making the same; pharmaceutical compositions comprising same; and methods of treating neurological diseases and disorders using same. In one aspect, the biocompatible copolymer can be poly(lactic-co-glycolic acid). In another aspect, the nanoparticle compositions can include one or more pharmaceutical agents including, but not limited to, antiretroviral agents. In still another aspect, the ionic liquids can include choline and an anion derived from a substituted or unsubstituted C2-C20 linear or branched fatty acid. In any of these aspects, the nanoparticle compositions are capable of crossing the blood brain barrier.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1B show Ionic liquid coats both empty poly (D,L-lactide-co-glycolide) 50:50 (PLGA) nanoparticles (NPs) and those loaded with 60 μg/mL abacavir. FIG. 1A: the size and FIG. 1B: surface charge of the bare empty poly(lactic-co-glycolic acid) (PLGA), Abacavir (ABC)-loaded PLGA, IL-coated empty PLGA, and ABC-loaded ionic liquid (IL)-coated PLGA. FIGS. 1C-1D show scanning electron microscopy (SEM) of Bare PLGA (FIG. 1C) and IL-coated PLGA (FIG. 1D), which shows morphological changes upon IL coating. Scale=1 μm.

FIGS. 2A-2G show IL-NPs dramatically enhance delivery to the brain in vivo and influence regional abacavir accumulation. FIGS. 2A-2D: Sprague-Dawley rat brain cross-sections shown after treatment with: (FIG. 2A) saline, (FIG. 2B) bare NPs loaded with DiD, or (FIG. 2C) IL-NPs loaded with DiD. Scale bar=1 mm. (FIG. 2D) Signal quantified by densitometry (area×mean intensity; n=1/group). FIG. 2E: Biodistribution (%) of injected 1,1′-Dioctadecyl-3,3,3′,3′-Tetramethylindocarbocyanine, 4-chlorobenzenesulfonate salt (DiD) in isolated organs (% ID organ, n=3/group; mean±SEM). †denotes significant difference from respective PLGA-DID-treated group; p<0.05 (two-tailed Student's t-test). FIGS. 2F-2G: Representative differences, by 1H-NMR spectroscopy, in abacavir (ABC) regional brain accumulation in Sprague-Dawley rat brains (n=3/group) post intra-carotid injection for (FIG. 2F) empty IL-PLGA NPs and (FIG. 2G) IL-PLGA NPs loaded with ABC. Key proton peak for ABC presence at 8.1 ppm is indicated (see red box, FIG. 2G).

FIGS. 3A-3D show IL-PLGA DID NPs colocalize selectively with microglia in vivo: a cross section of rat brain treated with IL-PLGA-1,1′-dioctadecyl-3,3,3′,3′-tetramethylindocarbocyanine, 4-chlorobenzenesulfonate salt (DiD), with differential staining for astrocytes (FIG. 3B) and microglia (FIG. 3C). Also shown is a nuclear stain (Hoechst) to demonstrate cytoarchitecture (FIG. 3A) and the DiD signal alone (FIG. 3D). Insets show IL-PLGA-DID co-localizes with microglia. (FIGS. 3A-3D) Colocalization of DiD-loaded IL-NPs (FIG. 3D) with microglia (FIG. 3C), but not astrocytes (FIG. 3B), is demonstrated in the parenchyma of the dorsal striatum (inset shows the head of the caudate across panels). Scale bar=1 mm.

FIGS. 4A-4E show a striatal blood vessel in coronal section. FIG. 4A: Hoechst nuclear stain with blood vessel outlined (FIG. 4B). FIG. 4C: Glial fibrillary acidic protein (GFAP)-labeled astrocytes. FIG. 4D: ionized calcium-binding adapter molecule 1 (Iba-1)-labeled microglia. FIG. 4E: IL-PLGA delivered DiD. Arrows point to DiD location across all images. Scale bar=50 μm. The IL is choline trans-2-hexenoate in a 1:2 molar ratio of cation to anion (choline trans-2-hexenoic acid or CA2HA, 1:2) and these data show that this IL mediates delivery not only through the blood brain barrier, but also colocalizes with HIV reservoir cells. Scale bar=50 microns.

FIGS. 5A-5D show colocalization of Hoechst nuclear stain (FIG. 5A) with ionized calcium-binding adapter molecule 1 (Iba-1) (FIGS. 5A-5B) and IL-PLGA-DID (FIGS. 5A and 5C). Somal expression of DiD is evident in microglia (FIGS. 5A-5C). (FIG. 5D) Fractional gated representation of FACS quantification (CDIIb+vs. CDIIb+DiD+) of isolated microglia show high colocalization with IL-PLGA DID NPs vs. saline background, which is CDIIb+ only (n=3 internal repetitions of n=1 brain extract/group±standard deviation). CC=corpus callosum, LV=lateral ventricle, AC=anterior commissure. Scale bar=10 microns in FIGS. 5A-5C.

FIG. 6A shows IL-PLGA NPs enter the brain by shearing through blood vessels and are uptaken by microglia in the caudate/putamen. FIG. 6A: Bare PLGA NPs were sporadically seen in endothelial cells. FIGS. 6B-6C: IL-PLGA NPs were seen in parenchymal microglia and around blood vessels in endothelial cells. (FIG. 6C) DiD signal from IL-coated NPs is observed in every microglial soma captured in the field and in several suspected endothelial cells surrounding apparent blood vessels. (FIGS. 6D-6E) Labeling with von Willebrand factor confirmed the presence of DiD in endothelial cells. (FIG. 6F) Z-stack imaging supports intracellular localization of DiD in microglia (see bottom and right orthogonal views for virtual cross-section). (FIGS. 6G-6H) DiD co-localization to microglia was frequently uniform next to large vessels. * Indicates blood vessel. Arrows localize DiD in FIGS. 6F-6H. Images collected using a 63×/NA1.4 oil lens. Scale bars=10 microns in every panel.

FIG. 7 shows IL-coated PLGA NPs encapsulate abacavir, suppress viral replication in HIV-1 treated human PBMCs without cytotoxicity, and show enhanced human microglia and human astrocyte uptake in vitro. CA2HA 1:2 coated PLGA NPs effectively deliver abacavir to human cells and suppress viral expression (indicated by quantification of the HIV capsid protein, p24). HIV-1_{BaL} viral replication (n=2) is attenuated by CA2HA 1:2-coated PLGA NPs loaded with abacavir (ABC; 60 μg/mL; n=3), ABC alone (n=3), or CA2HA 1:2-coated empty PLGA NPs (n=3). *indicates significant difference from mock-infected cells (n=2); \hat{ }indicates significant difference from HIV-infected cells; p<0.05 (Repeated-Measures ANOVA)

FIGS. 8A-8Dii show cell viability of uninfected and HIV-1 infected cells under various treatment conditions. HIV treatment alone induced ~20% cell death. This was obviated by the addition of any treatment used. IL-PLGA with ABC was less cytotoxic than any other treatment tested. FIG. 8A: Bare and coated PLGA NPs with ABC (60 μg/mL) show little cytotoxicity when compared to mock-infected PBMCs. *indicates significant difference from mock-infected cells; *indicates significant difference from mock-infected cells; \hat{ }indicates significant difference from HIV-1 infected cells; p<0.05 (Repeated Measures ANOVA). FIGS. 8Bi-8Dii: Immunocytochemistry on co-cultured primary human astrocytes and primary human microglia were performed: FIGS. 8Bi-8Bii: media-control, FIGS. 8Ci-8Cii: PLGA NPs loaded with DiD, and FIGS. 8Di-8Dii: IL-PLGA NPs loaded with DiD. Cells were co-labeled with anti-Iba-1 or -GFAP and Hoechst nuclear stain. Intracellular DiD accumulation was qualitatively greater when PLGA-NPs were coated with IL (see FIGS. 8Di-8Dii). Scale=50 μm.

FIG. 9 shows average encapsulation efficiency for ABC of different nanoparticle compositions.

FIGS. 10A-10G show ¹H-NMR spectroscopy affirms CA2HA 1:2 IL coating on PLGA NPs and verifies presence of abacavir (ABC) ART when encapsulated inside IL-PLGA NPs. (FIG. 10A) ¹H NMR of choline and trans-2-hexenoic acid (CA2HA 1:2). (FIG. 10B) Intact PLGA NPs, (FIG. 10C) Intact IL-PLGA ABC NPs, zoomed-in baseline of intact (FIG. 10D) PLGA ABC and (FIG. 10E) IL-PLGA ABC NPs, and (FIG. 10F) destroyed IL-PLGA NPs revealing abacavir proton peaks, with 0.5 mg/mL abacavir drug reference in (FIG. 10G). (FIG. 10E) includes 20 μL TMS and (FIG. 10F) includes 2 μL TMS in D₂O (500 μL~2 mg/mL), respectively, for IL and ABC quantification.

FIGS. 11A-11C show FACS detects only trace CA2HA 1:2 DID PLGA NPs circulating on isolated RBCs from whole blood at the in vivo 6-hour timepoint, indicating NPs have sheared into blood-filtering organs and is secondary verification of first-pass accumulation into the brain. Pictured, left column: singlet-gated side vs. forward scattering profile of isolated RBCs from whole Sprague Dawley rat blood and right column: gated singlet RBCs vs. far-red fluorescence, from whole rat blood intravenously infused with (FIG. 11A) 0.9% saline (no fluorescence), (FIG. 11B) bare PLGA DID NPs (no fluorescence), and (FIG. 11C) CA2HA 1:2-coated PLGA DID NPs (trace fluorescence).

FIG. 12 shows scanning electron microscopy of CA2HA 1:2 DID PLGA NP on mouse red blood cells (in vitro after fluorescence assisted cell sorting; top row) and after 24 hours (bottom row). After 24 hours, shear marks are apparent on the cells and few nanoparticles remain.

FIG. 13 shows hemolysis kinetics for DiD encapsulated CA2HA 1:2 PLGA nanoparticles at 405 nm (n=4). Hemolysis rates remained low for up to 2 h for both CAHA 1:2 DiD and bare PLGA DID NPs.

FIG. 14 shows an exemplary ionic liquid synthesis of choline and trans-2-hexenoic acid in a 1:2 ratio according to one embodiment of the present disclosure.

FIG. 15 shows an exemplary method for synthesizing IL coated NPs according to one embodiment of the present disclosure. 1 mL of 1 mg/mL PLGA in acetonitrile containing a dye (e.g. DiD) or an HIV drug (ABC) is placed in 3 mL deionized water and shaken at 1200 rpm for 3 h. 1 drop of ionic liquid is added and the resultant mixture is shaken at 800 rpm for 2 h. The solution is then centrifuged at 2500 rpm for 1 h at 4° C. The nanoparticle solution is brought up to 1 mL total volume in buffer and stored at 4° C.

FIGS. 16A-16B show transmission electron microscopy of (FIG. 16A) bare PLGA NPs (left) and (FIG. 16B) ionic liquid-coated PLGA NPs (right), while FIGS. 16C-16D show bare and IL-coated PLGA NPs, respectively, by scanning electron microscopy.

FIG. 17 shows a mechanism for delivery of antiretroviral therapy (ART) according to one embodiment of the present disclosure. IL-PLGA-NPs loaded with ART are taken up by the red blood cells, mechanically sheared from the surfaces of the red blood cells, taken up by infected microglia, and ART is released into the microglia cytoplasm by ester bond cleavage.

FIG. 18 shows CA2HA 1:2 NPs colocalize with mouse BALB/c endothelial junctions in lungs 24 h after tail-vein injection (IV). 150+ slice z-stack (Confocal Microscopy). Showing: vascular endothelial cadherin, Von-Willebrand Factor, DAPI, and DID IL-PLGA NPs.

FIGS. 19A-19B show encapsulation efficiency (EE), UV-Visible absorption spectra, and fluorescent emission spectra verify far-red DiD encapsulation in and comparable fluorescence between bare PLGA and IL-coated NPs. Fluorescence read across 200 μL NPs or Org. Phase/well). When normalized to the DiD organic phase used for synthesis (100%), CA2HA 1:2-coated PLGA DID NPs yield an EE of 60.43±2.03% (n=3). Bare PLGA DID NPs yield an EE of 65.09±2.29% (n=3). (FIG. 19A) By UV-Vis., the two-phase absorbance (shoulder peak at 600 nm & characteristic peak at 640 nm) of DiD dye (red & black) slightly shifts as a result of molecular packing inside of both 1 mg/mL bare and IL-coated PLGA NPs in USP-grade saline. The shoulder peak at 600 nm can be attributed to additional electronic transitions, vibrational modes, or formation of aggregates/excimers. Interestingly, CA2HA 1:2 further enhances the absorbance at 600 nm, compared to bare PLGA NPs, suggesting an additional compositional phase shift on the PLGA surface (where covalent dye interactions persist) upon IL coating. (FIG. 19B) The apex of the DiD fluorescent emission spectra (670 nm) is preserved from the 2% (wt/vol) organic phase to the encapsulated bare and IL-coated PLGA NPs, with an emission intensity trend comparable to EE via fluorescent plate reader. Interestingly, there is a noticeable red-shift observed when DID is present together with PLGA in ACN solvent suggestive of the expected covalent binding between the dye and polymer, creating a new conformation of packed dye molecules around the polymer which is then encapsulated during nanoprecipitation.

FIGS. 20A-20H show FACS of purified microglia isolated from CA2HA 1:2 DID PLGA NP (FIGS. 20A-20D) and saline (FIGS. 20E=20H)-treated Sprague Dawley whole rat brains verifies microglia-selective uptake at the in vivo 6-hour timepoint, verifying confocal microscopy of frozen brain sections. Pictured, left to right: side vs. forward scattering of isolated rat brain microglia, gated microglia singlet populations, FITC-CDIIb+ positive forward scattering microglia, and FITC-CDIIb+ microglia colocalized against far-red fluorescence (DiD).

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

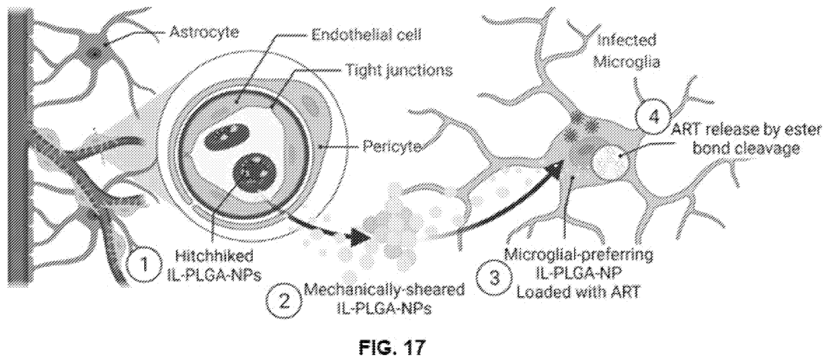

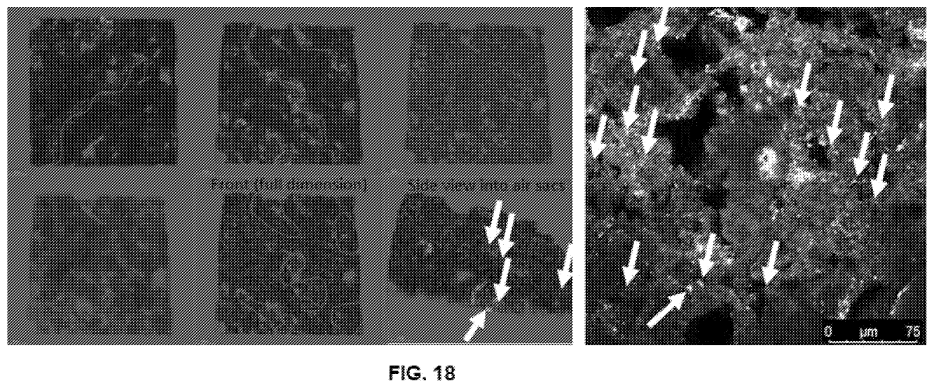

Disclosed herein is a method for transporting pharmaceuticals across the blood brain barrier. In an aspect, ionic liquid-coated nanoparticles adhere to the surface of red blood cells, are later mechanically-sheared from the red blood cells, and able to enter microglia, astrocytes, and other brain cells, thus crossing the blood brain barrier (see FIG. 17), where the nanoparticles can deliver pharmaceutical agents including, but not limited to, antiretroviral therapy effective against neuro-HIV.

### Nanoparticle Compositions

In one aspect, disclosed herein is a nanoparticle composition. In a further aspect, the nanoparticle composition includes at least a plurality of nanoparticles. In a still further aspect, each nanoparticle of the plurality includes a core, wherein the core is made from or includes a biocompatible copolymer. In another aspect, each nanoparticle of the plurality further includes an ionic liquid coating surrounding the core. In any of these aspects, the nanoparticle composition is capable of crossing the blood brain barrier by selective binding of the ionic liquid coating to red blood cell membranes. In an aspect, the biocompatible copolymer is or includes poly(lactic-co-glycolic acid), polycaprolactone-polyamidoamine (PCL-PAMAM), or any combination thereof. In still another aspect, the ionic liquid is made from choline and an anion derived from a saturated or unsaturated C2-C20 linear or branched fatty acid.

In some aspects, in the disclosed nanoparticle compositions, after crossing the blood brain barrier, the nanoparticle composition localizes in one or more cell types selected from red blood cells, monocyte-derived macrophages, microglia, perivascular macrophages, and astrocytes.

In another aspect, the fatty acid can be a saturated fatty acid, a monounsaturated fatty acid, a polyunsaturated fatty acid, or any combination thereof, and the anion can be selected from butanoate, 2-butenoate, 3-butenoate, pentanoate, 2-pentenoate, 3-pentenoate, hexanoate, 2-hexenoate, 3-hexenoate, trans-2-methyl-2-pentenoate, heptanoate, 2-heptenoate, 3-heptenoate, octanoate, 2-octenoate, 3-octenoate, nonanoate, 2-nonenoate, 3-nonenoate, decanoate, 2-decenoate, 3-decenoate, undecanoate, 2-undecenoate, dodecanoate, fumarate, malonate, maleate, malate, acetoxyacetate, ethoxyacetate, 3-mercaptopropionate, or any combination thereof. In one aspect, the anion is 2-hexenoate.

In any of these aspects, the molar ratio of the choline to the fatty acid is from about 1:1 to about 1:4, or from about 1:1 to about 1:2, or is 1:1, 1:1.5, 1:2, 1:2.5, 1:3, 1:3.5, or 1:4, or a combination of any of the foregoing values, or a range encompassing any of the foregoing values.

In another aspect, the nanoparticle composition further includes a pharmaceutical agent. In one aspect, the pharmaceutical agent can be present at from about 0.2% to about 10%, from about 0.5% to about 5%, or at about 2% (w/v) relative to a total volume of the nanoparticle composition. In some aspects, the pharmaceutical agent can be an anti-retroviral agent such as, for example, abacavir, emtricitabine, lamivudine, tenofovir disoproxil fumarate, zidovudine, doravirine, efavirenz, etravirine, nevirapine, rilpivirine, atazanavir, darunavir, fosamprenavir, ritonavir, tipranavir, enfuvirtide, maraviroc, cabotegravir, dolutegravir, raltegravir, fostemsavir, ibalizumab-uiyk, cobicistat, or any combination thereof.

In yet another aspect, the disclosed nanoparticle composition can further include at least one pharmaceutically-acceptable carrier or excipient. In any of these aspects, the nanoparticle composition can be biocompatible.

In one aspect, in the disclosed compositions, each nanoparticle of the plurality can have an average diameter of from about 65 nm to about 215 nm, from about 65 to about 205 nm, about 175 to about 205 nm, or of about 190 nm, or can have an average diameter of 65, 70, 75, 80, 85, 90, 95, 100, 105, 110, 115, 120, 125, 130, 135, 140, 145, 150, 155, 160, 165, 170, 175, 180, 185, 190, 195, 200, or about 205 nm, or a combination of any of the foregoing values, or a range encompassing any of the foregoing values. In another aspect, each nanoparticle of the plurality can have a surface charge of from about −35 mV to about −65 mV, from about −45 mV to about −65 mV, or of from about −50 to about −60 mv, or of about −55 mV, or can have a surface charge of about −35, −40, −45, −50, −55, −60, or about −65 mV, or a combination of any of the foregoing values, or a range encompassing any of the foregoing values. In any of these aspects, the plurality of nanoparticles can have a polydispersity index of less than about 0.3, or can be substantially monodisperse.

### Methods for Treating Neurological Diseases and Disorders

Also disclosed herein is a method for treating a neurological disease or disorder in a subject, the method including at least the step of administering a disclosed nanoparticle composition to the subject. In one aspect, the subject can be a mammal such as, for example, a human, cat, dog, horse, cattle, sheep, goat, hamster, guinea pig, rabbit, mouse, or rat, or the subject can be a bird such as, for example, a chicken, turkey, duck, goose, or parrot.

In another aspect, in the disclosed method, the ionic liquid or composition can be administered intravenously. Further in this aspect, the site of administration can be the carotid artery (e.g. intracarotid administration). In another aspect, from about 0.1 mg to about 5 mg, or from about 0.25 mg to about 2 mg, or about 0.1, 0.25, 0.5, 0.71, 1, 1.5, 2, 2.5, 3, 3.5, 4, 4.5, or about 5 mg of nanoparticles can be administered per kg of subject body weight. In yet another aspect, the nanoparticle composition persists in the subject for about 48 hours or less, or for about 24 hours or less.

In a further aspect, the neurological disease or disorder comprises neuroHIV, depression, addiction, migraine, schizophrenia, Alzheimer's disease, dementia, brain cancer, a lysosomal storage disease, traumatic brain injury, or any combination thereof. In some aspects, the nanoparticles accumulate in at least one organ or cell type associated with a neuroHIV reservoir. In a further aspect, the organ can be the brain, spleen, liver, bone marrow, thymus, lungs, lymph nodes, small intestine, colon, genital tract, or any combination thereof. In a further aspect, the cell type can be or include microglia.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by,” “comprising,” “comprises,” “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a pharmaceutical agent,” “a neurological disorder,” or “an ionic liquid,” include, but are not limited to, mixtures or combinations of two or more such pharmaceutical agents, neurological disorders, or ionic liquids, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y.’ The range can also be expressed as an upper limit, e.g. ‘about x, y, z, or less’ and should be interpreted to include the specific ranges of ‘about x,’ ‘about y’, and ‘about z’ as well as the ranges of ‘less than x’, less than y′, and ‘less than z’. Likewise, the phrase ‘about x, y, z, or greater’ should be interpreted to include the specific ranges of ‘about x,’ ‘about y,’ and ‘about z’ as well as the ranges of ‘greater than x,’ greater than y,’ and ‘greater than z.’ In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a PLGA polymer refers to an amount that is sufficient to achieve the desired improvement in the property modulated by the formulation component, e.g. achieving the desired particle diameter, encapsulation efficiency of pharmaceutical agents, or the like. The specific level in terms of wt % in a composition required as an effective amount will depend upon a variety of factors including the amount and type of lactide and glycolide monomer units, amount and type of pharmaceutical agent to be encapsulated, amount and identity of ionic liquid coating, and disease or condition being targeted with the nanoparticle composition.

As used herein, the terms “optional” or “optionally” means that the subsequently described event or circumstance can or cannot occur, and that the description includes instances where said event or circumstance occurs and instances where it does not.

“Biocompatible” as used herein refers to a compound or composition, such as, for example, an ionic liquid or nanoparticle composition, that does not damage or harm living tissue in a subject. In one aspect, a biocompatible material does not kill any living cells or trigger an immune response in a subject when the compound or composition is administered or applied to the subject. In one aspect, the nanoparticle compositions disclosed herein are biocompatible.

As used herein, “administering” can refer to an administration intravenous. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “therapeutic agent” or “pharmaceutical agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

In various aspects, a disclosed intravenous pharmaceutical composition can include pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions, or suspensions, including saline and buffered media. Parenteral vehicles can include mannitol, normal serum albumin, sodium chloride solution, Ringer's dextrose, dextrose and sodium chloride, lactated Ringer's, and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases, and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

Unless otherwise specified, pressures referred to herein are based on atmospheric pressure (i.e. one atmosphere).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### ASPECTS

The present disclosure can be described in accordance with the following numbered Aspects, which should not be confused with the claims.

Aspect 1. A nanoparticle composition comprising a plurality of nanoparticles, each nanoparticle of the plurality comprising a core, wherein the core comprises a biocompatible copolymer, and each nanoparticle of the plurality further comprising an ionic liquid coating surrounding the core, wherein the nanoparticle composition is capable of crossing the blood brain barrier by selective binding of the ionic liquid coating to red blood cell membranes;
wherein the biocompatible copolymer comprises poly(lactic-co-glycolic acid), polycaprolactone-polyamidoamine (PCL-PAMAM), or any combination thereof; and
wherein the ionic liquid comprises choline and an anion derived from a saturated or unsaturated C2-C20 linear or branched fatty acid.

Aspect 2. The nanoparticle composition of aspect 1, wherein after crossing the blood brain barrier, the nanoparticle composition localizes in one or more cell types selected from red blood cells, monocyte-derived macrophages, microglia, perivascular macrophages, and astrocytes.

Aspect 3. The nanoparticle composition of aspect 1 or 2, wherein the fatty acid comprises a saturated fatty acid, a monounsaturated fatty acid, a polyunsaturated fatty acid, or any combination thereof.

Aspect 4. The nanoparticle composition of any one of aspects 1-3, wherein the anion comprises butanoate, 2-butenoate, 3-butenoate, pentanoate, 2-pentenoate, 3-pentenoate, hexanoate, 2-hexenoate, 3-hexenoate, trans-2-methyl-2-pentenoate, heptanoate, 2-heptenoate, 3-heptenoate, octanoate, 2-octenoate, 3-octenoate, nonanoate, 2-nonenoate, 3-nonenoate, decanoate, 2-decenoate, 3-decenoate, undecanoate, 2-undecenoate, dodecanoate, fumarate, malonate, maleate, malate, acetoxyacetate, ethoxyacetate, 3-mercaptopropionate, or any combination thereof.

Aspect 5. The nanoparticle composition of aspect 4, wherein the anion comprises 2-hexenoate.

Aspect 6. The nanoparticle composition of any one of aspects 1-5, wherein a molar ratio of the choline to the fatty acid is from about 1:1 to about 1:4.

Aspect 7. The nanoparticle composition of aspect 6, wherein a molar ratio of the choline to the fatty acid is about 1:2.

Aspect 8. The nanoparticle composition of any one of aspects 1-7, further comprising a pharmaceutical agent.

Aspect 9. The nanoparticle composition of aspect 8, wherein the pharmaceutical agent is present at from about 0.2 to about 10% (w/v) relative to a total volume of the nanoparticle composition.

Aspect 10. The nanoparticle composition of aspect 9, wherein the pharmaceutical agent is present at about 2% (w/v) relative to a total volume of the nanoparticle composition.

Aspect 11. The nanoparticle composition of any one of aspects 8-10, wherein the pharmaceutical agent comprises an anti-retroviral agent.

Aspect 12. The nanoparticle composition of aspect 11, wherein the antiretroviral agent comprises abacavir, emtricitabine, lamivudine, tenofovir disoproxil fumarate, zidovudine, doravirine, efavirenz, etravirine, nevirapine, rilpivirine, atazanavir, darunavir, fosamprenavir, ritonavir, tipranavir, enfuvirtide, maraviroc, cabotegravir, dolutegravir, raltegravir, fostemsavir, ibalizumab-uiyk, cobicistat, or any combination thereof.

Aspect 13. The nanoparticle composition of aspect 12, wherein the antiretroviral agent is abacavir.

Aspect 14. The nanoparticle composition of any one of aspects 1-13, further comprising at least one pharmaceutically-acceptable carrier or excipient.

Aspect 15. The nanoparticle composition of any one of aspects 1-14, wherein the nanoparticle composition is biocompatible.

Aspect 16. The nanoparticle composition of any one of aspects 1-15, wherein each nanoparticle of the plurality has an average diameter of from about 65 nm to about 215 nm.

Aspect 17. The nanoparticle composition of any one of aspects 1-16, wherein each nanoparticle of the plurality has a surface charge of from about-35 mV to about-65 mV.

Aspect 18. The nanoparticle composition of any one of aspects 1-17, wherein the plurality of nanoparticles have a polydispersity index of less than about 0.3.

Aspect 19. The nanoparticle composition of any one of aspects 1-18, wherein the plurality of nanoparticles are substantially monodisperse.

Aspect 20. A method for treating a neurological disease or disorder in a subject, the method comprising administering the nanoparticle composition of any one of aspects 1-19 to the subject.

Aspect 21. The method of aspect 20, wherein the subject is a mammal or bird.

Aspect 22. The method of aspect 21, wherein the mammal is a human, cat, dog, horse, cattle, sheep, goat, hamster, guinea pig, rabbit, mouse, or rat.

Aspect 23. The method of aspect 21, wherein the bird is a chicken, turkey, duck, goose, or parrot.

Aspect 24. The method of any one of aspects 20-23, wherein the composition is administered intravenously.

Aspect 25. The method of aspect 24, wherein a site of administration is intracarotid.

Aspect 26. The method of any one of aspects 20-25, wherein from about 0.1 mg to about 5 mg of nanoparticles are administered per kg of subject body weight.

Aspect 27. The method of any one of aspects 20-26, wherein the nanoparticle composition persists in the subject for about 48 hours or less.

Aspect 28. The method of aspect 27, wherein the nanoparticle composition persists in the subject for about 24 hours or less.

Aspect 29. The method of any one of aspects 20-28, wherein the neurological disease or disorder comprises neuroHIV, depression, addiction, migraine, schizophrenia, Alzheimer's disease, dementia, brain cancer, a lysosomal storage disease, traumatic brain injury, or any combination thereof.

Aspect 30. The method of any one of aspects 20-29, wherein the nanoparticles accumulate in at least one cell type or organ associated with a neuroHIV reservoir.

Aspect 31. The method of aspect 30, wherein the organ comprises the brain, spleen, liver, bone marrow, thymus, lungs, lymph nodes, small intestine, colon, genital tract, or any combination thereof.

Aspect 32. The method of aspect 30, wherein the at least one cell type comprises microglia.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Materials and Methods

### Materials

Choline bicarbonate cation (Sigma-Aldrich, #C7519-500ML) and trans-2-hexenoic acid anion (Sigma-Aldrich, #W316903-1 KG-K) were obtained from Sigma Aldrich. Neat synthesized ionic liquid (IL) was characterized by ¹H NMR (400 MHz Bruker Ascend) in DMSO-D6 (99.96 atom % D) (Sigma-Aldrich, #156914-10) and by Karl Fischer Titration for water content (Metrohm Coulometer #899). Resomer® RG 504 H, Poly (D, L-lactide-co-glycolide) 50:50 (PLGA) (acid terminated) was obtained from Sigma Aldrich (#719900). To study biodistribution of the NPs, the hydrophobic far-red dye 1,1′-Dioctadecyl-3,3,3′,3′-Tetramethylindocarbocyanine, 4-chlorobenzenesulfonate salt solid (DiD) was used (ThermoFisher, #D7757), while HIV drug cargo encapsulation was performed with abacavir sulfate (ABC) (Cayman Chemical, #14746). HPLC-grade Acetonitrile (Sigma-Aldrich, #34851-4) was used as the organic phase of nanoprecipitation, with the aqueous phase consisting of Ultrapure MilliQ water (#Milli-Q IQ 7000). After cold nanoprecipitation and solvent evaporation in a 100 mL round bottom flask (Sigma-Aldrich, #Z414492) on a 9-plate magnetic stir plate, NPs were cold-centrifuge filtered (Thermo Scientific Sorvall Ultracentrifuge, #ST8R) using a 30 kD MWCO Amicon Ultra-4 filter (EMD Millipore, #UFC803024). For IL-PLGA NP NMR quantification and analysis, Deuterium Oxide (D2O) (99 atom % D) solvent was used as the aqueous phase (Sigma-Aldrich, #435767-1 KG). Neat tetramethylsilane (TMS) (density=0.648 g/mL, MW 88.22 g/mol) NMR internal standard for relative IL and ABC drug quantification was obtained from Sigma Aldrich (#87920-25ML). For NP physiological use, 1× Phosphate-Buffered Saline (PBS, pH 7.4.) was obtained from Gibco™ (#10010072), and 0.9% USP-injection-grade saline from Fisher Scientific (#NC9054335). Homogenization studies were performed with an IKA 5G homogenizer (IKA, #0003304000), 50% BioXtra Trichloroacetic acid (Sigma-Aldrich, #T9159-500G), 5M NaOH (Sigma-Aldrich, #221465-500G), 1×Tris-Buffered Saline (TBS) (Cell Signaling, #12498), and Fisherbrand PH meter (Accumet AB150). Pierce RIPA Lysis Extraction Buffer was acquired from Thermo Scientific (#89901). Triton-X (Cat #807423) was obtained from BM biomedicals LLC, 0.1% BSA (Cat #A6003) from Sigma), trypan blue (Cat #15250-061) from Gibco, and BSA (Cat #A6003) from Sigma. anti Iba 1, Rabbit, (Cat #019-19741) was obtained from Wako Chemicals USA Inc. Anti-Glial Fibrillary Acidic Protein, Clone GA5. Alexa Fluor 488 goat anti mouse IgG, (Cat #A11002) and Texas Red Goat anti-Rabbit IgG (Cat #T2767), and Hoechst (Cat #33342) were sourced from Invitrogen. Goat Serum (Cat #D204-00-0050) was obtained from Rockland Inc. Condition media contained RPMI 1640 (Cat #22400-105), 10% FBS heat-inactivated (Cat #SH30910.03), 1% Pen/Strep, (Cat #15140-122), 1% L-Glutamine (Cat #17-605E) from Fisher Scientific. Human Astrocyte Primary Cell culture (Cat #36058-01) and Human Microglia Primary Cell culture (Cat #37089-01) were sourced from Celprogen. PBMC cells (Cat #SER-PBMC—P-F. Lot PBMC070721ABCDE) were obtained from Zen Bio Inc. HIV-1 virus was obtained from the NIH AIDS reagent program, HIV-1 Ba-L virus, Cat #510, Lot #150058. p24 antigen assay (ELISA, Cat #5447) was obtained from ABL Inc.

### Ionic Liquid Synthesis and Characterization

Choline 2-hexenoate (choline trans-2-hexenoic acid, CA2HA 1:2) was synthesized as previously published. Briefly, choline bicarbonate (80% in water) was combined dropwise with 98% pure trans-2-hexenoic acid at a 1:2 molar ionic ratio at 40° C. stirring in an oil bath overnight. CA2HA 1:2 IL was then rotary evaporated to remove any residual solvent at 15 mbar for 2 hours at 60° C., and then vacuum dried at −760 mmHg for 48 hours at 60° C. The product (332.5 g/mol, density 1.66 g/mL) was massed by analytical balance to calculate yield (82.6%), evaluated by Karl Fischer to measure IL water content (1.68% (wt/wt)), and assayed by NMR to evaluate purity and chemical identity: ¹H NMR (300 MHz, DMSO) δ 6.41-6.29 (m, 2H), 5.50 (dt, J=15.6, 1.5 Hz, 2H), 3.62 (dq, J=5.0, 2.6 Hz, 2H), 3.20 (dd, J=6.3, 3.7 Hz, 2H), 2.89 (s, 9H), 1.83-1.72 (m, 4H), 1.10 (p, J=7.3 Hz, 4H), 0.58 (t, J=7.3 Hz, 6H).

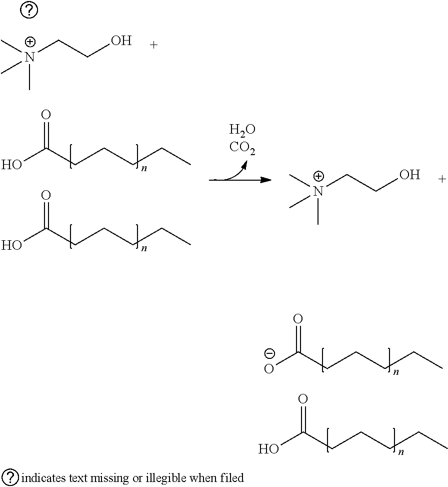

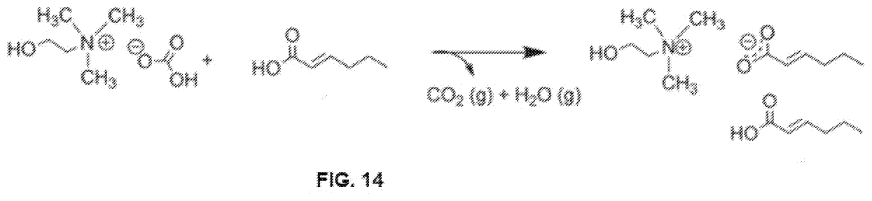

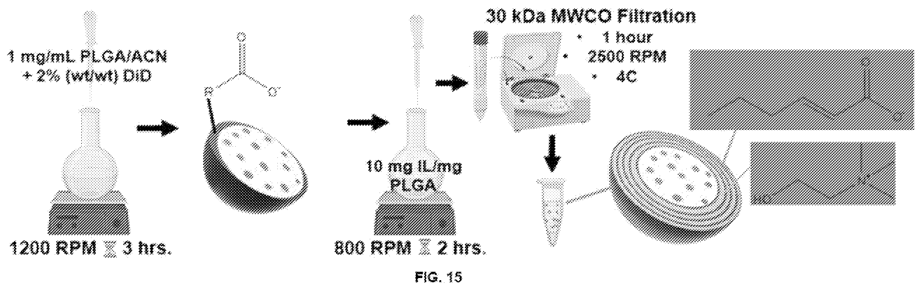

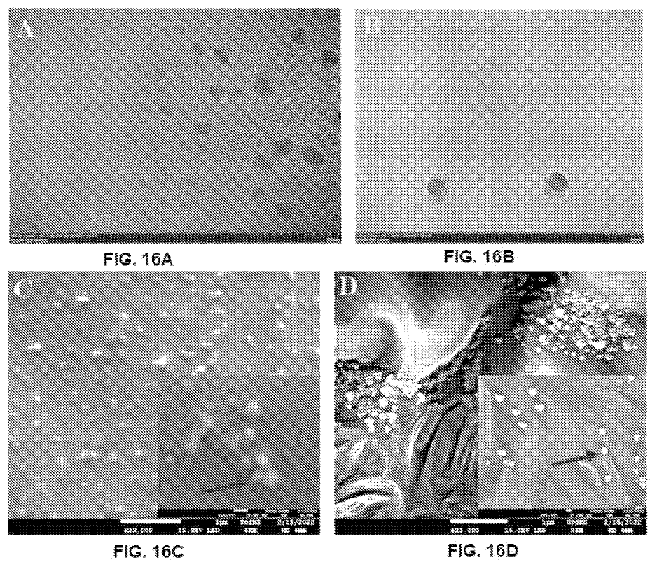

Scheme 1 shows representative ionic liquid synthesis. Salt metathesis reaction of choline bicarbonate and carboxylic acid in a 1:2 molar ratio to form cholinium carboxylate 1:2 illustrates the extraneous production of water and carbon dioxide (see also FIG. 14).

Neat synthesized ionic liquid (IL) was characterized by ¹H NMR (400 MHz Bruker Ascend) in DMSO-d6 (99.96 atom % D) and by Karl Fischer titration for water content (Metrohm Coulometer from Metrohm, Herisau, Switzerland).

Choline Butanoate 1:1 (CABA 1:1) ¹H NMR (400 MHz, d6-DMSO) δ 3.89-3.81 (m, 2H), 3.45 (t, J=5.0 Hz, 2H), 3.15 (s, 9H), 1.86 (t, J=7.0 Hz, 2H), 1.42 (h, J=7.4 Hz, 2H), 0.81 (t, J=7.3 Hz, 3H).

Choline Butanoate 1:2 (CABA 1:2) ¹H NMR (400 MHz, d6-DMSO) δ 3.84 (dq, J=5.4, 2.6 Hz, 2H), 3.42 (dq, J=4.7, 2.6 Hz, 2H), 3.12 (s, 9H), 2.04 (ddd, J=7.8, 4.9, 3.0 Hz, 4H), 1.46 (h, J=7.3 Hz, 4H), 0.84 (td, J=7.4, 1.7 Hz, 6H).

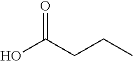

Choline 2-Butenoate 1:1 (CA2BE 1:1) ¹H NMR (400 MHz, d6-DMSO) δ 6.30 (dtd, J=13.7, 6.9, 3.0 Hz, 1H), 5.67 (dd, J=15.2, 2.1 Hz, 2H), 3.83 (q, J=4.1 Hz, 2H), 3.49-3.42 (m, 2H), 3.15 (s, 9H), 1.66 (d, J=7.0 Hz, 3H). Nanoparticles coated with this ionic liquid were shown to successfully hitchhike red blood cells.

Choline 2-Butenoate 1:2 (CA2BE 1:2) ¹H NMR (400 MHz, d6-DMSO) δ 6.55 (ddq, J=16.1, 7.0, 2.9 Hz, 2H), 5.78 (dd, J=15.6, 2.3 Hz, 2H), 3.85 (dq, J=5.6, 2.7 Hz, 2H), 3.44 (tt, J=5.1, 2.6 Hz, 2H), 3.14 (d, J=1.8 Hz, 9H), 1.74 (dd, J=6.9, 1.9 Hz, 6H).

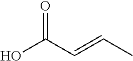

Choline 3-Butenoate 1:1 (CA3BE 1:1) ¹H NMR (400 MHz, d6-DMSO) δ 5.93 (ddt, J=17.3, 9.7, 7.3 Hz, 1H), 4.90-4.79 (m, 2H), 3.85 (h, J=2.6 Hz, 2H), 3.43 (dd, J=6.1, 3.8 Hz, 2H), 3.13 (s, 9H), 1.66 (dd, J=6.7, 1.8 Hz, 1H).

Choline 3-Butenoate 1:2 (CA3BE 1:2) ¹H NMR (400 MHz, d6-DMSO) δ 6.55 (dtd, J=13.7, 8.7, 7.7, 5.9 Hz, 1H), 6.02-5.69 (m, 2H), 5.02-4.89 (m, 2H), 3.84 (dq, J=5.4, 2.6 Hz, 2H), 3.46-3.37 (m, 2H), 3.12 (s, 9H), 1.75 (dd, J=6.9, 1.7 Hz, 4H).

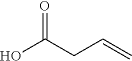

Choline Pentanoate 1:1 (CAPA 1:1) ¹H NMR (400 MHz, d6-DMSO) δ 3.85 (dq, J=8.0, 2.8 Hz, 2H), 3.46-3.34 (m, 2H), 3.13 (s, 9H), 1.91-1.81 (m, 2H), 1.39 (tt, J=7.8, 6.5 Hz, 2H), 1.29-1.15 (m, 2H), 0.83 (t, J=7.3 Hz, 3H).

Choline Pentanoate 1:2 (CAPA 1:2) ¹H NMR (400 MHz, d6-DMSO) δ 3.89-3.80 (m, 2H), 3.45-3.38 (m, 2H), 3.12 (s, 9H), 2.01 (t, J=7.4 Hz, 4H), 1.48-1.36 (m, 4H), 1.31-1.17 (m, 4H), 0.84 (t, J=7.3 Hz, 6H). Nanoparticles coated with this ionic liquid were shown to successfully hitchhike red blood cells.

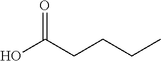

Choline 2-Pentenoate 1:1 (CA2PE 1:1) ¹H NMR (400 MHz, d6-DMSO) δ 6.34 (dtd, J=15.8, 6.4, 2.9 Hz, 1H), 5.63 (dd, J=15.5, 1.7 Hz, 1H), 3.84 (dq, J=5.9, 2.7 Hz, 2H), 3.46 (dd, J=6.2, 3.9 Hz, 2H), 3.15 (s, 9H), 2.08-1.96 (m, 2H), 0.93 (td, J=7.4, 2.0 Hz, 3H).

Choline 2-Pentenoate 1:2 (CA2PE 1:2) ¹H NMR (400 MHz, d6-DMSO) δ 6.61 (dt, J=15.3, 6.2 Hz, 2H), 5.75 (dt, J=15.5, 1.7 Hz, 2H), 3.85 (dq, J=5.4, 2.6 Hz, 2H), 3.44 (dd, J=6.1, 4.1 Hz, 2H), 3.14 (s, 9H), 2.10 (tt, J=9.1, 6.6 Hz, 4H), 0.97 (t, J=7.5 Hz, 6H).

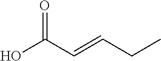

Choline 3-Pentenoate 1:1 (CA3PE 1:1) ¹H NMR (400 MHz, d6-DMSO) δ 5.58 (dt, J=14.1, 6.8 Hz, 1H), 5.32 (dq, J=13.3, 6.3 Hz, 1H), 3.89 (dq, J=5.8, 2.7 Hz, 2H), 3.51 (dd, J=6.5, 3.6 Hz, 2H), 3.21 (d, J=2.5 Hz, 9H), 2.72 (d, J=7.3 Hz, 2H), 1.63 (d, J=6.7 Hz, 3H).

Choline 3-Pentenoate 1:2 (CA3PE 1:2) ¹H NMR (400 MHz, d6-DMSO) δ 5.51 (dtd, J=13.9, 7.0, 1.7 Hz, 2H), 5.43-5.30 (m, 2H), 3.84 (td, J=5.6, 2.5 Hz, 2H), 3.44 (dt, J=7.1, 3.4 Hz, 2H), 3.13 (d, J=3.2 Hz, 9H), 2.80 (d, J=7.2 Hz, 4H), 1.60 (d, J=6.8 Hz, 6H). Nanoparticles coated with this ionic liquid were shown to successfully hitchhike red blood cells.

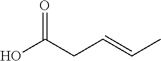

Choline Hexanoate 1:1 (CAHA 1:1) ¹H NMR (400 MHz, d6-DMSO) δ 3.84 (dt, J=7.4, 2.7 Hz, 2H), 3.49-3.42 (m, 2H), 3.16 (s, 9H), 1.87 (t, J=7.5 Hz, 2H), 1.41 (p, J=7.4 Hz, 2H), 1.27-1.15 (m, 4H), 0.83 (td, J=7.1, 1.8 Hz, 3H).

Choline Hexanoate 1:2 (CAHA 1:2) ¹H NMR (400 MHz, d6-DMSO) δ 3.84 (td, J=5.6, 2.6 Hz, 2H), 3.44 (dq, J=7.5, 3.7 Hz, 2H), 3.14 (dd, J=5.9, 2.8 Hz, 9H), 2.05 (dt, J=9.1, 4.5 Hz, 4H), 1.45 (p, J=7.3 Hz, 4H), 1.23 (pd, J=8.4, 7.9, 2.9 Hz, 8H), 0.88-0.80 (m, 6H).

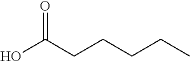

Choline 2-Hexenoate 1:1 (CA2HA 1:1) ¹H NMR (400 MHz, d6-DMSO) δ 6.27 (dt, J=15.4, 7.0 Hz, 1H), 5.62 (dd, J=15.4, 1.6 Hz, 1H), 3.89-3.81 (m, 2H), 3.50-3.43 (m, 2H), 3.15 (s, 9H), 1.99 (qd, J=7.1, 1.5 Hz, 2H), 1.35 (h, J=7.3 Hz, 2H), 0.86 (t, J=7.4 Hz, 3H). Nanoparticles coated with this ionic liquid were shown to successfully hitchhike red blood cells.

Choline 2-Hexenoate 1:2 (CA2HA 1:2) ¹H NMR (400 MHz, d6-DMSO) δ 6.39-6.27 (m, 2H), 5.51 (d, J=15.5 Hz, 2H), 3.60 (dt, J=7.5, 3.5 Hz, 2H), 3.21 (dt, J=9.4, 5.2 Hz, 2H), 2.90 (d, J=6.4 Hz, 9H), 1.81 (q, J=7.3 Hz, 4H), 1.18-1.09 (m, 4H), 0.61 (td, J=7.4, 3.0 Hz, 6H). Nanoparticles coated with this ionic liquid were shown to successfully hitchhike red blood cells.

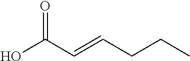

Choline 3-Hexenoate 1:1 (CA3HA 1:1) ¹H NMR (400 MHz, d6-DMSO) δ 5.56-5.48 (m, 1H), 5.29 (ddd, J=15.3, 7.2, 5.5 Hz, 1H), 3.84 (dq, J=5.6, 2.7 Hz, 2H), 3.49-3.42 (m, 2H), 3.15 (s, 9H), 2.67-2.61 (m, 2H), 1.94 (p, J=7.2 Hz, 2H), 0.91 (t, J=7.5 Hz, 3H). Nanoparticles coated with this ionic liquid were shown to successfully hitchhike red blood cells.

Choline 3-Hexenoate 1:2 (CA3HA 1:2) ¹H NMR (400 MHz, d6-DMSO) δ 5.56-5.48 (m, 1H), 5.29 (ddd, J=15.3, 7.2, 5.5 Hz, 1H), 3.84 (dq, J=5.6, 2.7 Hz, 2H), 3.49-3.42 (m, 2H), 3.15 (s, 9H), 2.67-2.61 (m, 2H), 1.94 (p, J=7.2 Hz, 2H), 0.91 (t, J=7.5 Hz, 3H).

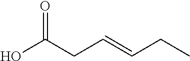

Choline trans-2-Methyl-2-Pentenoate 1:1 (CAGAS 1:1) ¹H NMR (400 MHz, d6-DMSO) δ 6.22 (td, J=7.3, 1.6 Hz, 1H), 3.85 (dq, J=7.7, 2.8 Hz, 2H), 3.49-3.42 (m, 2H), 3.15 (s, 9H), 1.98 (p, J=7.5 Hz, 2H), 1.64 (d, J=1.7 Hz, 3H), 0.91 (t, J=7.6 Hz, 3H).

Choline trans-2-Methyl-2-Pentenoate 1:2 (CAGAS 1:2) ¹H NMR (400 MHz, d6-DMSO) δ 6.43 (td, J=7.4, 1.8 Hz, 2H), 3.86 (td, J=5.6, 2.6 Hz, 2H), 3.49-3.40 (m, 2H), 3.15 (s, 9H), 2.06 (p, J=7.5 Hz, 4H), 1.69 (d, J=1.8 Hz, 6H), 0.95 (t, J=7.6 Hz, 6H). Nanoparticles coated with this ionic liquid were shown to successfully hitchhike red blood cells.

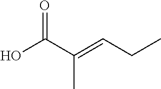

Choline Heptanoate 1:1 (CAHPA 1:1) ¹H NMR (400 MHz, d6-DMSO) δ 3.85 (dt, J=7.2, 2.7 Hz, 2H), 3.44-3.40 (m, 2H), 3.12 (s, 9H), 1.83 (td, J=7.5, 2.0 Hz, 2H), 1.39 (p, J=7.3 Hz, 2H), 1.20 (dt, J=7.9, 3.1 Hz, 5H), 0.85 (t, J=6.9 Hz, 3H). Nanoparticles coated with this ionic liquid were shown to successfully hitchhike red blood cells.

Choline Heptanoate 1:2 (CAHPA 1:2) ¹H NMR (400 MHz, d6-DMSO) δ 3.84 (dq, J=5.4, 2.7 Hz, 2H), 3.43-3.38 (m, 2H), 3.11 (s, 9H), 2.02 (ddd, J=7.4, 5.9, 1.5 Hz, 4H), 1.42 (d, J=7.3 Hz, 5H), 1.31-1.17 (m, 13H), 0.85 (t, J=6.8 Hz, 6H).

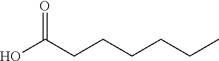

Choline 2-Heptenoate 1:1 (CA2HPE 1:1) ¹H NMR (400 MHz, d6-DMSO) δ 6.24 (dt, J=15.4, 6.9 Hz, 1H), 5.60 (dt, J=15.2, 1.5 Hz, 1H), 3.86 (td, J=5.6, 2.7 Hz, 2H), 3.49-3.42 (m, 2H), 3.15 (s, 9H), 2.06-1.96 (m, 2H), 1.30 (dtt, J=17.9, 7.4, 4.0 Hz, 4H), 0.86 (t, J=6.9 Hz, 3H).

Choline 2-Heptenoate 1:2 (CA2HPE 1:2) ¹H NMR (400 MHz, d6-DMSO) δ 6.60 (dt, J=15.7, 7.0 Hz, 2H), 5.81 (dt, J=15.4, 1.5 Hz, 2H), 3.92 (dq, J=5.5, 2.7 Hz, 2H), 3.54-3.47 (m, 2H), 3.20 (s, 9H), 2.15 (qd, J=7.0, 1.6 Hz, 4H), 1.49-1.29 (m, 8H), 0.94 (t, J=7.1 Hz, 6H).

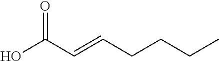

Choline 3-Heptenoate 1:1 (CA3HPE 1:1) ¹H NMR (400 MHz, d6-DMSO) δ 5.62-5.48 (m, 1H), 5.27-5.14 (m, 1H), 3.85 (dq, J=5.5, 2.7 Hz, 2H), 3.47-3.40 (m, 3H), 3.13 (s, 9H), 2.58 (dd, J=7.1, 1.5 Hz, 2H), 1.91 (q, J=7.5 Hz, 2H), 1.32 (h, J=7.4 Hz, 2H), 0.86 (t, J=7.3 Hz, 3H).

Choline 3-Heptenoate 1:2 (CA3HPE 1:2) ¹H NMR (400 MHz, d6-DMSO) δ 5.57-5.45 (m, 2H), 5.39-5.27 (m, 2H), 3.85 (td, J=5.6, 2.8 Hz, 2H), 3.45-3.38 (m, 2H), 3.12 (s, 9H), 2.75 (dd, J=7.0, 1.4 Hz, 3H), 1.93 (q, J=7.1 Hz, 3H), 1.33 (h, J=7.3 Hz, 4H), 0.86 (t, J=7.3 Hz, 6H).

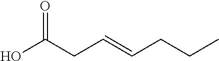

Choline Octanoate 1:1 (CAOA 1:1) ¹H NMR (400 MHz, d6-DMSO) δ 3.85 (dq, J=8.1, 2.8 Hz, 2H), 3.46-3.39 (m, 6H), 3.13 (s, 9H), 1.82 (t, J=7.4 Hz, 2H), 1.40 (q, J=7.3 Hz, 2H), 1.23 (qd, J=12.4, 11.2, 7.1 Hz, 9H), 0.86 (t, J=6.9 Hz, 3H).

Choline Octanoate 1:2 (CAOA 1:2) ¹H NMR (400 MHz, d6-DMSO) δ 3.65-3.56 (m, 2H), 3.25-3.18 (m, 2H), 2.90 (s, 9H), 1.80 (t, J=7.5 Hz, 4H), 1.20 (t, J=7.3 Hz, 4H), 1.03-0.95 (m, 16H), 0.59 (t, J=6.7 Hz, 6H).

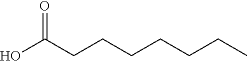

Choline 2-Octenoate 1:1 (CA2OE 1:1) ¹H NMR (400 MHz, d6-DMSO) δ 6.28 (dt, J=15.4, 6.9 Hz, 1H), 5.67-5.58 (m, 1H), 3.85 (dq, J=5.4, 2.6 Hz, 2H), 3.50-3.43 (m, 2H), 3.15 (s, 9H), 2.00 (qd, J=7.1, 1.5 Hz, 2H), 1.34 (p, J=7.1 Hz, 3H), 1.25 (tt, J=8.1, 5.1 Hz, 4H), 0.85 (t, J=6.9 Hz, 3H).

Choline 2-Octenoate 1:2 (CA2OE 1:2) ¹H NMR (300 MHz, d6-DMSO) δ 6.62-6.46 (m, 2H), 5.74 (dt, J=15.5, 1.6 Hz, 2H), 3.91-3.79 (m, 2H), 3.43 (dd, J=6.1, 4.0 Hz, 2H), 3.13 (s, 9H), 2.08 (qd, J=7.1, 1.5 Hz, 4H), 1.41-1.23 (m, 12H), 0.86 (t, J=6.7 Hz, 6H).

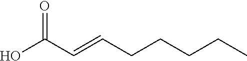

Choline 3-Octenoate 1:1 (CA3OE 1:1) ¹H NMR (400 MHz, d6-DMSO) δ 5.59-5.47 (m, 1H), 5.17 (dt, J=14.7, 6.7 Hz, 1H), 3.83 (dd, J=6.2, 3.7 Hz, 2H), 3.11 (s, 9H), 2.09 (s, 2H), 1.92 (d, J=6.9 Hz, 2H), 1.28 (q, J=5.1, 3.4 Hz, 4H), 0.90-0.82 (m, 3H).

Choline 3-Octenoate 1:2 (CA3OE 1:2) ¹H NMR (400 MHz, d6-DMSO) δ 5.50 (dt, J=14.5, 6.9 Hz, 2H), 5.35 (dt, J=15.3, 6.7 Hz, 2H), 3.84 (dq, J=5.7, 2.8 Hz, 2H), 3.44 (dd, J=7.6, 4.1 Hz, 2H), 3.17-3.11 (m, 9H), 2.80 (t, J=5.6 Hz, 4H), 1.95 (q, J=6.8 Hz, 4H), 1.27 (dq, J=11.3, 7.7, 5.5 Hz, 8H), 0.85 (td, J=7.2, 3.0 Hz, 6H).

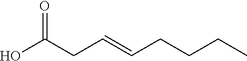

Choline Nonanoate 1:1 (CANA 1:1) ¹H NMR (400 MHz, d6-DMSO) δ 3.84 (dq, J=5.4, 2.6 Hz, 2H), 3.48-3.41 (m, 2H), 3.14 (s, 9H), 1.88 (t, J=7.5 Hz, 2H), 1.40 (t, J=7.2 Hz, 2H), 1.22 (q, J=6.1, 5.2 Hz, 10H), 0.85 (t, J=6.7 Hz, 3H).

Choline Nonanoate 1:2 (CANA 1:2) ¹H NMR (400 MHz, d6-DMSO) δ 3.89-3.80 (m, 2H), 3.45-3.37 (m, 2H), 3.12 (s, 9H), 2.02 (t, J=7.4 Hz, 4H), 1.43 (p, J=7.0 Hz, 5H), 1.23 (d, J=3.8 Hz, 22H), 0.90-0.81 (m, 6H).

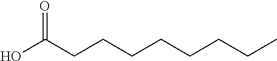

Choline 2-Nonenoate 1:1 (CA2NE 1:1) ¹H NMR (400 MHz, d6-DMSO) δ 6.23 (dt, J=15.7, 6.9 Hz, 1H), 5.60 (dd, J=15.3, 1.5 Hz, 1H), 3.86 (dq, J=7.8, 2.8 Hz, 2H), 3.50-3.43 (m, 2H), 3.16 (s, 9H), 2.00 (qd, J=7.1, 1.5 Hz, 2H), 1.33 (t, J=6.8 Hz, 2H), 1.26 (q, J=4.7 Hz, 6H), 0.90-0.82 (m, 3H).

Choline 2-Nonenoate 1:2 (CA2NE 1:2) ¹H NMR (400 MHz, d6-DMSO) δ 6.53 (dtd, J=17.6, 7.4, 3.0 Hz, 2H), 5.74 (dd, J=15.5, 1.7 Hz, 2H), 3.90-3.81 (m, 2H), 3.47-3.38 (m, 2H), 3.13 (d, J=2.1 Hz, 9H), 2.08 (qd, J=7.1, 1.5 Hz, 4H), 1.36 (q, J=7.1 Hz, 4H), 1.32-1.23 (m, 12H), 0.90-0.82 (m, 6H).

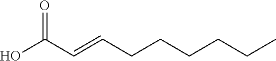

Choline 3-Nonenoate 1:1 (CA3NE 1:1) ¹H NMR (400 MHz, d6-DMSO) δ 5.54 (dtd, J=15.6, 7.1, 1.4 Hz, 1H), 5.28-5.16 (m, 1H), 3.84 (dq, J=5.5, 2.7 Hz, 2H), 3.47-3.40 (m, 2H), 3.14 (s, 9H), 2.59 (dd, J=7.1, 1.5 Hz, 2H), 1.92 (q, J=6.9 Hz, 2H), 1.36-1.18 (m, 6H), 0.86 (t, J=6.7 Hz, 3H).

Choline 3-Nonenoate 1:2 (CA3NE 1:2) ¹H NMR (400 MHz, d6-DMSO) δ 5.50 (dtd, J=15.1, 6.8, 1.3 Hz, 2H), 5.36 (ddd, J=15.3, 7.3, 5.9 Hz, 2H), 3.84 (dq, J=5.5, 2.6 Hz, 2H), 3.46-3.39 (m, 2H), 3.12 (s, 9H), 2.79 (dd, J=6.7, 1.5 Hz, 4H), 1.95 (q, J=6.9 Hz, 4H), 1.38-1.26 (m, 8H), 1.24 (d, J=6.4 Hz, 4H), 0.86 (t, J=6.8 Hz, 6H).

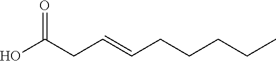

Choline Decanoate 1:1 (CADA 1:1) ¹H NMR (400 MHz, d6-DMSO) δ 3.87-3.81 (m, 2H), 3.46 (t, J=4.9 Hz, 2H), 3.16 (s, 9H), 1.90 (t, J=7.6 Hz, 2H), 1.40 (t, J=7.3 Hz, 2H), 1.20 (s, 12H), 0.82 (t, J=6.6 Hz, 3H).

Choline Decanoate 1:2 (CADA 1:2) ¹H NMR (400 MHz, d6-DMSO) δ 3.91 (dq, J=5.4, 2.6 Hz, 2H), 3.54-3.47 (m, 2H), 3.20 (s, 9H), 2.11 (t, J=7.5 Hz, 4H), 1.51 (p, J=7.0 Hz, 4H), 1.34 (s, 3H), 1.30 (s, 20H), 0.92 (t, J=6.8 Hz, 6H).

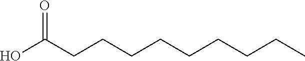

Choline 2-Decenoate 1:1 (CA2DE 1:1) ¹H NMR (400 MHz, d6-DMSO) δ 6.37 (dt, J=15.5, 6.9 Hz, 1H), 5.71 (d, J=15.3 Hz, 1H), 3.94 (dq, J=5.8, 2.6 Hz, 2H), 3.58-3.51 (m, 2H), 3.24 (s, 9H), 2.09 (q, J=7.1 Hz, 2H), 1.48-1.34 (m, 6H), 1.34 (s, 4H), 0.94 (t, J=6.8 Hz, 3H).

Choline 2-Decenoate 1:2 (CA2DE 1:2) ¹H NMR (400 MHz, d6-DMSO) δ 6.52 (dt, J=15.8, 6.9 Hz, 2H), 5.77-5.69 (m, 2H), 3.85 (p, J=2.8 Hz, 2H), 3.47-3.40 (m, 2H), 3.13 (s, 9H), 2.07 (q, J=7.1 Hz, 4H), 1.37 (p, J=6.8 Hz, 4H), 1.29 (d, J=8.3 Hz, 4H), 1.25 (s, 12H), 0.86 (t, J=6.6 Hz, 6H).

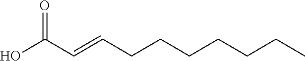

Choline 3-Decenoate 1:1 (CA3DE 1:1) ¹H NMR (400 MHz, d6-DMSO) δ 5.57-5.45 (m, 1H), 5.31-5.19 (m, 1H), 3.88-3.80 (m, 3H), 3.44-3.37 (m, 3H), 3.11 (s, 9H), 2.64 (dd, J=7.0, 1.4 Hz, 2H), 1.93 (q, J=6.6 Hz, 2H), 1.35-1.18 (m, 11H), 1.01-0.92 (m, 1H), 0.86 (h, J=3.1 Hz, 3H).

Choline 3-Decenoate 1:2 (CA3DE 1:2) ¹H NMR (400 MHz, d6-DMSO) δ 5.49 (dtt, J=15.1, 6.8, 1.3 Hz, 2H), 5.34 (dtt, J=14.9, 6.7, 1.4 Hz, 2H), 3.88-3.80 (m, 2H), 3.44-3.37 (m, 2H), 3.11 (s, 9H), 2.77 (dd, J=6.8, 1.3 Hz, 4H), 1.95 (q, J=6.4 Hz, 4H), 1.36-1.22 (m, 17H), 0.90-0.82 (m, 6H).

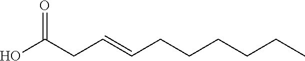

Choline Undecanoate 1:1 (CAUA 1:1) ¹H NMR (400 MHz, d6-DMSO) δ 3.95 (dq, J=5.8, 2.7 Hz, 2H), 3.60-3.53 (m, 2H), 3.26 (s, 9H), 1.99 (t, J=7.5 Hz, 2H), 1.55-1.47 (m, 2H), 1.32 (d, J=4.5 Hz, 14H), 0.94 (t, J=6.7 Hz, 3H).

Choline Undecanoate 1:2 (CAUA 1:2) ¹H NMR (400 MHz, d6-DMSO) δ 3.64-3.57 (m, 2H), 3.20 (d, J=4.8 Hz, 2H), 2.90 (s, 9H), 1.79 (t, J=7.5 Hz, 4H), 1.21 (q, J=7.0 Hz, 4H), 1.01 (s, 4H), 0.97 (s, 24H), 0.59 (t, J=6.7 Hz, 6H).

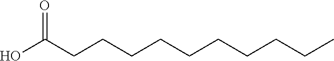

Choline 2-Undecenoate 1:1 (CA2UE 1:1) ¹H NMR (400 MHz, d6-DMSO) δ 6.36 (dt, J=14.5, 6.9 Hz, 1H), 5.71 (d, J=15.3 Hz, 1H), 4.00-3.91 (m, 2H), 3.65-3.55 (m, 2H), 3.28 (s, 9H), 2.09 (q, J=7.1 Hz, 2H), 1.47-1.35 (m, 6H), 1.34 (s, 6H), 0.94 (t, J=6.7 Hz, 3H).

Choline 2-Undecenoate 1:2 (CA2UE 1:2) ¹H NMR (400 MHz, d6-DMSO) δ 6.50 (dt, J=15.6, 6.9 Hz, 2H), 5.73 (dd, J=15.5, 1.7 Hz, 2H), 3.86 (dq, J=5.6, 2.8 Hz, 2H), 3.46 (dd, J=5.9, 4.1 Hz, 2H), 3.15 (s, 9H), 2.06 (q, J=7.2 Hz, 4H), 1.36 (p, J=6.9 Hz, 4H), 1.25 (q, J=6.5, 4.1 Hz, 20H), 0.85 (t, J=6.8 Hz, 6H).

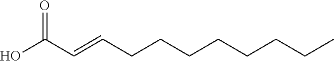

Choline Dodecanoate 1:1 (CADDA 1:1) ¹H NMR (400 MHz, d6-DMSO) δ 3.85 (q, J=2.9 Hz, 2H), 3.44 (dd, J=6.1, 3.8 Hz, 2H), 3.14 (s, 9H), 1.85 (t, J=7.5 Hz, 2H), 1.40 (p, J=7.1 Hz, 2H), 1.22 (d, J=8.4 Hz, 16H), 0.85 (t, J=6.6 Hz, 3H).

Choline Dodecanoate 1:2 (CADDA 1:2) ¹H NMR (400 MHz, d6-DMSO) δ 3.81 (dq, J=5.6, 2.7 Hz, 2H), 3.40 (dd, J=5.8, 4.2 Hz, 2H), 3.10 (s, 9H), 2.00 (t, J=7.5 Hz, 4H), 1.41 (q, J=7.1 Hz, 4H), 1.18 (s, 32H), 0.80 (t, J=6.7 Hz, 6H).

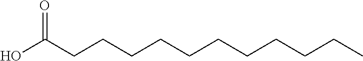

Choline Fumarate 1:1 (CAFU 1:1) ¹H NMR (400 MHz, d6-DMSO) δ 6.46 (s, 2H), 3.84 (dq, J=5.3, 2.6 Hz, 2H), 3.48-3.37 (m, 3H), 3.12 (s, 9H).

Choline Fumarate 1:2 (CAFU 1:2) ¹H NMR (400 MHz, d6-DMSO) δ 6.55 (s, 4H), 3.84 (dq, J=5.3, 2.6 Hz, 2H), 3.41 (dd, J=6.0, 4.1 Hz, 2H), 3.11 (s, 9H).

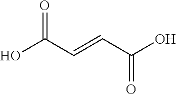

Choline Malonate 1:1 (CAMA 1:1) ¹H NMR (400 MHz, d6-DMSO) δ 3.84 (td, J=5.7, 2.7 Hz, 2H), 3.41 (t, J=5.2 Hz, 2H), 3.12 (s, 9H), 2.77 (s, 2H).

Choline Malonate 1:2 (CAMA 1:2) ¹H NMR (400 MHz, d6-DMSO) δ 3.84 (td, J=5.8, 2.7 Hz, 2H), 3.44-3.36 (m, 2H), 3.13 (s, 9H), 2.93 (s, 4H).

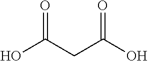

Choline Maleate 1:1 (CAME 1:1) ¹H NMR (400 MHz, d6-DMSO) δ 6.12 (d, J=2.6 Hz, 2H), 3.86 (td, J=5.7, 2.7 Hz, 2H), 3.43 (t, J=5.3 Hz, 2H), 3.13 (s, 9H).

Choline Maleate 1:2 (CAME 1:2) ¹H NMR (400 MHz, d6-DMSO) δ 6.16 (s, 4H), 3.84 (dq, J=5.3, 2.6 Hz, 2H), 3.47-3.35 (m, 2H), 3.13 (s, 9H).

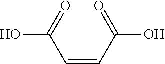

Choline Malate 1:1 (CAMI 1:1) ¹H NMR (400 MHz, d6-DMSO) δ 7.07 (s, 1H), 3.93 (dd, J=9.1, 4.6 Hz, 1H), 3.84 (td, J=5.7, 2.7 Hz, 2H), 3.45-3.38 (m, 2H), 3.11 (s, 9H), 2.55 (dd, J=15.6, 9.1 Hz, 1H), 2.31 (dd, J=15.5, 4.6 Hz, 1H).

Choline Malate 1:2 (CAMI 1:2) ¹H NMR (400 MHz, d6-DMSO) δ 7.87 (s, 2H), 4.16 (dd, J=7.1, 5.6 Hz, 2H), 3.82 (dq, J=7.7, 2.6 Hz, 2H), 3.45-3.34 (m, 2H), 3.08 (d, J=8.1 Hz, 9H), 2.61 (dd, J=15.6, 5.5 Hz, 2H), 2.39 (dd, J=15.6, 7.2 Hz, 2H).

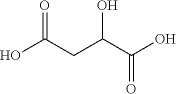

Choline Acetoxyacetate 1:1 (CAAA 1:1) ¹H NMR (400 MHz, d6-DMSO) δ 4.16 (s, 2H), 3.83 (h, J=2.6 Hz, 2H), 3.47-3.37 (m, 2H), 3.13 (s, 9H), 1.99 (s, 3H).

Choline Acetoxyacetate 1:2 (CAAA 1:2) ¹H NMR (400 MHz, d6-DMSO) δ 4.30 (s, 4H), 3.76 (s, 2H), 3.42 (s, 2H), 3.11 (s, 9H), 2.03 (s, 6H).

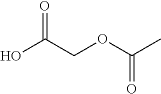

Choline Ethoxyacetate 1:1 (CAEA 1:1) ¹H NMR (400 MHz, d6-DMSO) δ 3.89-3.82 (m, 2H), 3.44 (s, 2H), 3.41 (s, 2H), 3.39 (s, 2H), 3.13 (s, 9H), 1.06 (t, J=7.0 Hz, 3H).

Choline Ethoxyacetate 1:2 (CAEA 1:2) ¹H NMR (400 MHz, d6-DMSO) δ 3.85 (dq, J=8.0, 2.8 Hz, 2H), 3.77 (s, 4H), 3.44 (d, J=7.2 Hz, 4H), 3.41 (d, J=7.0 Hz, 2H), 3.14 (s, 9H), 1.07 (t, J=7.1 Hz, 6H).

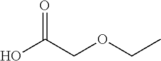

Choline 3-Mercaptopropionate 1:1 (CA3MP 1:1) ¹H NMR (400 MHz, d6-DMSO) δ 3.82 (dq, J=7.8, 2.8 Hz, 2H), 3.43 (dt, J=5.0, 3.3 Hz, 2H), 3.13 (d, J=4.0 Hz, 9H), 2.53 (t, J=7.1 Hz, 2H), 2.21 (t, J=7.1 Hz, 2H).

Choline 3-Mercaptopropionate 1:2 (CA3MP 1:2) ¹H NMR (400 MHz, d6-DMSO) δ 3.84 (s, 2H), 3.45-3.34 (m, 2H), 3.12 (d, J=4.7 Hz, 9H), 2.58 (dd, J=8.6, 4.9 Hz, 4H), 2.39 (q, J=6.9, 6.3 Hz, 4H).

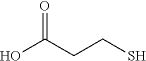

Choline Trans-3-Methyl-Trans-2-Pentenoate 1:1 (CA3ME2PE 1:1) ¹H NMR (400 MHz, DMSO) δ 5.54-5.46 (m, 1H), 3.98-3.90 (m, 2H), 3.55-3.48 (m, 10H), 3.21 (s, 9H), 2.04-1.94 (m, 3H), 1.89-1.43 (m, 2H), 1.00 (dt, J=15.0, 7.5 Hz, 3H). Nanoparticles coated with this ionic liquid were shown to successfully hitchhike red blood cells.

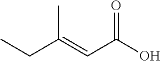

Choline Trans-3-Methyl-Trans-2-Hexenoate 1:1 (CA3ME2HA 1:1) ¹H NMR (400 MHz, DMSO) δ 5.57-5.52 (m, 1H), 4.14 (s, 1H), 3.98-3.90 (m, 3H), 3.56-3.49 (m, 2H), 3.23 (s, 9H), 2.03-1.93 (m, 5H), 1.47 (h, J=7.4 Hz, 2H), 0.92 (t, J=7.3 Hz, 3H). Nanoparticles coated with this ionic liquid were shown to successfully hitchhike red blood cells.

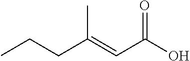

### Nanoparticle Synthesis and Characterization

NP Synthesis. PLGA and IL-PLGA NPs were synthesized as previously published. Briefly, a stock solution of 1 mg/ml DiD/ACN was prepared and combined with a stock solution of 1 mg/mL PLGA/ACN at 2% by weight of the polymer. The organic phase was then combined dropwise in a 100 mL round bottom flask at 1200 RPM with an aqueous phase of either 3 mL Milli-Q water or D20 water (for chemical characterization) and allowed to stir on a magnetic plate for 3 hours in the dark at 25° C. 10 mg neat IL/mg PLGA was then added to the solution in the form of one liquid drop in the center of the stirring vortex, and allowed to stir for 2 more hours at 25° C. in the dark, before 30 kDa MWCO centrifuge filtration at 4° C. and 2500 RPM for 1 hour. Bare PLGA NPs were filtered more gently between 1500-2000 RPM. The filtrate was then collected and resuspended to 1 mg/mL in either Milli-Q water, D20, 1×PBS, or 0.9% USP-grade saline for the respective characterization or biological application. Samples were stored at 4° C. in the dark for up to 2 weeks, although used within the first week of synthesis.

NP synthesis with Abacavir (ABC) cargo: PLGA and IL-PLGA NPs were synthesized again as above, however with a different preparation of the HIV drug cargo. A stock of abacavir (ABC) was prepared in ACN at a 1 mg/mL concentration and kept cold at −20° C. The drug stock was then combined with the organic phase again at 2% (wt ABC/wt PLGA) however scaled respectively by molecular weight of ABC (286.3 g/mol) vs. DiD (1052.08 g/mol). After vortexing to incorporate, the organic phase was then stored at −20° C. for 2 hours before use. When performing nanoprecipitation, round bottom flasks on the magnetic stir plate were surrounded by reusable ice blocks to ensure a cold environment for the stirring solution. After capping, both PLGA and IL-PLGA NPs were filtered as described above and stored at 4° C. for the same time frame.

NP Size and Surface Charge Characterization: For both 1 mg/ml filtered DiD and ABC-encapsulated PLGA NPs, a Malvern Zetasizer pro blue was used (software version 3.0) for Dynamic Light Scattering (DLS) measurements. An optimized calibration time of 40 seconds was used for both samples after a 120 second optimization time was used to evaluate the scattering profile of ABC-encapsulated NPs, with a fluorescent filter engaged for DID NPs. A dip cell (Malvern ZEN1002) was used for zeta potential surface measurements while a DTS 0012 polystyrene cuvette was used for size determination. All samples were evaluated, at minimum, in triplicate.

Cargo Encapsulation Efficiency (EE): To validate that the fluorescence of the tracked NPs in vivo was derived from cargo encapsulation and not from free DiD dye leakage, encapsulation efficiency, absorption by UV-Visible spectroscopy, and fluorescence emission were measured, as described below.

Relative Encapsulated Fluorescence (DiD): In a 96-well black opaque plate, 200 μL of synthesized 2% DiD 1 mg/mL PLGA and CA2HA 1:2 coated PLGA NPs were plated per independently synthesized replicate (n=3) and compared to 200 μL buffer used for suspension and 200 μL of the same 2% Organic Phase stock used to synthesize the resulting particles. The plate was read from the top and height adjusted to optimize for the volume of samples in the well, and then fluorescently measured at 640 nm excitation and 670 nm emission. Fluorescence of the buffer was subtracted from all samples, and then samples were divided by fluorescence of the 2% Organic stock (100%). Standard deviation was used to measure the spread of DiD encapsulation among the particles.

DiD-Nanoparticle Absorbance by UV-Vis Spectroscopy: Using Hellma Herasil quartz absorption cuvettes (standard cells, parameter 260-2,500 nm spectral range, pathlength 10 mm, chamber volume 3,500 μL), the UV-Vis absorption spectrum profile of 1 mg/ml of bare PLGA DiD NPs & CA2HA 1:2 PLGA DID NPs in 0.9% NaCl were measured from 200-800 nm, alongside controls of 0.02 mg DiD in ACN (free dye in solvent) & 0.02 mg DiD in 1 mg/mL PLGA: ACN organic phase (covalently-bound DiD-PLGA in ACN). The absorption spectra of the prepared nanoparticles (and reference solutions) were measured using a double reference Cary 5000 spectrophotometer (Cary 5000 UV-Vis-NIR, Agilent). ACN and NaCl solutions were respectively used as reference account for any possible background absorbance and baseline correction was done to remove the same from interfering with the samples. The absorption intensity of each sample was normalized to compare the absorption profile of CA2HA 1:2 PLGA DID NPs against PLGA DID NPs and DiD dye to see the effect of IL coating on the absorption features of the DiD-loaded PLGA NPs.

DiD-Nanoparticle Fluorescent Emission by Fluorimetry: The fluorescence emission profile of 1 mg/ml of bare PLGA DiD NPs & CA2HA 1:2 PLGA DID NPs in 0.9% NaCl were measured on a Horiba FluoroMax SpectroFluorimeter (System #3657, model QM-8075-21-C with PTI ASOC-10) from the far-red to NIR region (650-800 nm), alongside controls of 0.02 mg DiD in ACN (free dye in solvent) & 0.02 mg DiD in 1 mg/mL PLGA: ACN organic phase (covalently-bound DiD-PLGA in ACN). The excitation wavelength for DiD was calibrated to 635.6 nm, with an excitation slit width resolution of 5 nm and an emission slit width of 5 nm in a quartz cuvette of path length 10 mm (step size: 0.5 nm, integration: 1 sec). The excitation wavelengths were generated by passing white light through a dual-grating system and the photons were collected through a photomultiplier tube. The fluorescence emission spectra of all the samples were exported as a. txt file, converted to .csv, and baseline subtracted by their respective solvent background and plotted in an overlay fashion for the better understanding of the trend of DiD emission profile among different DID NP samples with respect to DiD stock.

Quantitative encapsulation of ABC (¹H NMR): PLGA and CA2HA 1:2 PLGA NPs were synthesized in deuterium oxide as described previously, and then resuspended to 500 μL (2 mg/mL) to quantitatively measure composition of the IL coating on the surface of the PLGA NPs with 20 μL Tetramethylsilane (TMS) internal quantification standard: ¹H NMR (400 MHz, D₂O) δ 6.85 (dt, J=15.8, 7.0 Hz, 27H), 5.85 (dt, J=15.7, 1.5 Hz, 28H), 4.78 (d, J=1.3 Hz, 7140H), 4.09-4.02 (m, 27H), 3.81-3.71 (m, 21H), 3.64 (dd, J=11.8, 4.5 Hz, 45H), 3.60-3.47 (m, 73H), 3.25 (s, 136H), 2.72 (d, J=1.3 Hz, 14964H), 2.19 (qd, J=7.2, 1.6 Hz, 69H), 1.56-1.46 (m, 47H), 0.91 (t, J=7.4 Hz, 96H), 0.00 (s, 12H).

To break open the NPs, bare PLGA NPs and IL-PLGA NPs were first sonicated at 60° C. for 30 minutes to destroy the outer IL coating integrity, evidenced by a phase separation of cloudiness in the IL-PLGA NPs. To next dissociate the IL coating from the polymer core, all particles were vortexed at 25° C. on the highest setting for 10 minutes (in 2-minute intervals). The particles were then stored at −80° C. for 2 minutes to protect the ABC drug while inducing a bulk phase separation, and then vortexed at the highest setting for 2 min at 25° C. Lastly, to extrude the abacavir from the polymer core, all NPs were 30 kDa MWCO centrifuge-filtered for 1 hour at 4500 RPM at 40° C. Filtrate smaller than 30 kDa was collected at the bottom of the tube and stored at −20° C., concentrated (2×) with D₂O and evaluated for presence of drug by ¹H NMR at 400 MHz at the highest scan rate (proton64).

As the appearance of drug peaks were very small compared to the IL, 2 μL of TMS was then used in particular to obtain a relative understanding of the size of the small emerging peaks. TMS at 0 ppm was then integrated to 12 protons and compared to a small new singlet peak at 8.5 ppm indicating emergence of a unique proton proximal to ABC's cyclic nitrogenous base structure. Detection of other scattered protons in the cyclic structures in between IL peaks, possibly indicates protective effects of the IL upon ester degradation of the polymer. Representation of encapsulated ABC dose was then calculated:
0.002 mL TMS (0.648 g/mL)=0.0013 g TMS
0.0013 g TMS/88.22 g/mol TMS=0.0000146 mol TMS
0.0000146 mol TMS (~0.03 integral ratio, 8.5 ppm & 0 ppm peak) ~4.38×10⁻⁷ mol ABC
4.38× 10-7 mol ABC (286.3 g/mol ABC) ~ (0.000125 g ABC/2) (ug total ~1 mg/mL) ~62.7 μg/mL ABC encapsulated

Scanning Electron Microscopy (SEM): Bare PLGA and CA2HA 1:2 PLGA NPs without cargo were synthesized to evaluate topological and morphological differences on the nano-scale. Briefly, nanoparticles were prepared as described above, and then 10 μL of respective solution was dropcast onto plasma-cleaned 9.5×9.5 mm cylinder aluminum SEM sample stubs (JEOL, #10-005110-50). Samples were allowed to dry overnight at 4° C. and ambient conditions in an airtight environment to prevent dust deposition. The following day, all samples were then sputter coated with Palladium to enhance imaging contrast (16.5 mm, 35 mA, 200 seconds). Imaging was performed on the same day with a JSM-7200 FLV Field-Emission Scanning Electron Microscope (FESEM).

HIV Expression and Cytotoxicity: 3 independent batches of bare and CA2HA 1:2-coated PLGA NPs were prepared with abacavir (ABC) as above but in sterile Milli-Q water and re-suspended to 1 mg/ml with sterile 1×PBS pH 7.4. As ~60 μg/mL of drug was estimated by quantitative NMR, PBMC cells were stimulated with 10 μg/ml of PHA for 4 days. Stimulated PBMC cells were harvested at 400×g, RT for 10 min. The cells at 2×10⁶ cells/mL were used for each experimental condition and infected with 1 ng/ml of HIV-1 Bal+ in presence of 3 ng/ml of IL-2 and 2 μg/ml of polybrene. PBMC cells were treated in the absence or presence of nanoparticles at 10 UM final concentration. An equivalent concentration of free ABC (10 UM) was used to treat PBMC cells in parallel with NPs. The treated PBMC cells were harvested at 3-, 7-, and 10-days post infection. The cell viability was determined by trypan blue at each harvesting day and an aliquot of condition media was saved for further analysis on p24 antigen assay. The PBMC cells were treated freshly with NPs or ABC at each time point except day 10. The p24 antigen assay was performed according to manufacture protocol on experimental condition media collected on 3, 7, 10 days post HIV-1 infection for quantification of HIV-1.

Immunohistochemistry: human astrocyte and human microglia were co-cultured at 25000 and 6000 cell density in 6 well plate respectively. The cells were maintained according to manufacturer's protocol (Celprogen Inc, Torrance, CA). The cells were treated with and without PLGA-DID NP, and IL-PLGA-DID NP at 10 UM final concentration for 24 hr. At the end of incubation, the cells were fixed with 4% PFA for 10 min (25° C.), and then permeabilized (0.1% Triton-X, 0.1% BSA in PBS) for 30 minutes at 25° C. The cells were washed 3× with PBS and then blocked with blocking buffer (1% BSA, 1% normal goat serum in PBS) for 1 hour at 25° C. The cells were probed with rabbit anti-Iba1 (for microglia) and mouse anti-GFAP (for astrocyte) both at 1/200 dilution, overnight at 4° C. The cells were washed 3× with 1×PBS and incubated with Alexa 488 goat anti-rabbit, Texas red goat anti-mouse secondary antibodies (1/500 dilution), and Hoechst for nuclear counterstain for 1 hour at 25° C. Intracellular DiD accumulation was visualized using a TI2-E motorized, inverted microscope (Ti2-S JS, Nikon instrument Inc.) and showed that uptake of DiD-loaded PLGA-NPs coated with IL was greater than DiD loaded PLGA-NPs without IL.

In-Vivo Studies: All animal experiments were carried out under the supervision of the University of Mississippi IACUC under protocol #21-004.

Injection Procedure: Female Sprague-Dawley rats (~75 days of age) with in-dwelling, intra-carotid catheters were purchased from Envigo (St. Louis, MO, USA). Rats were acclimated to a temperature- and humidity-controlled room in the vivarium at the University of Mississippi for at least 7 d prior to experimental manipulation. Catheter patency was maintained via a flush (50% heparin in saline (IU/mL), 72 hrs. after arrival). At the time of manipulation, rats received a slow intravenous infusion of 500 μL over 5 min (100 μL/min) of saline, PLGA-NPs encapsulating DiD (1 mg/mL concentration in 0.9% (wt/vol) USP-grade saline), IL-PLGA-NPs encapsulating DiD (1 mg/mL concentration in 0.9% (wt/vol) USP-grade saline), or IL-PLGA NPs (with and without 60 μg/mL ABC) in 0.9% (wt/vol) USP-grade saline.

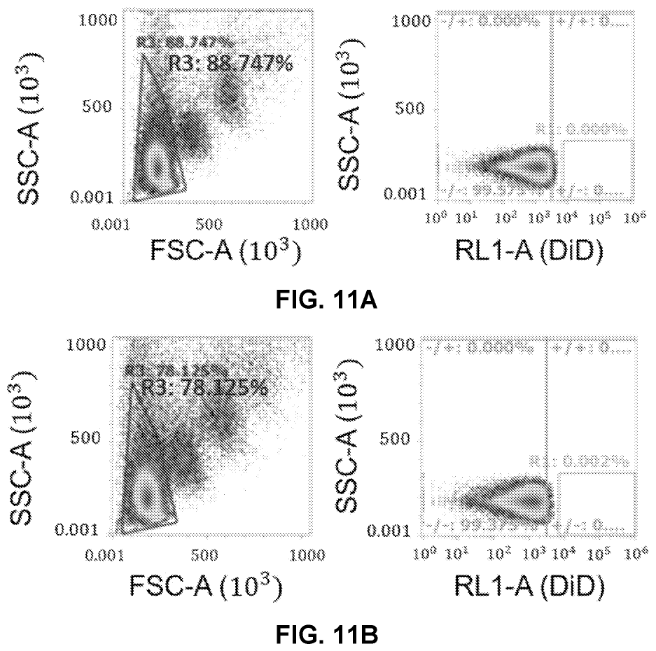

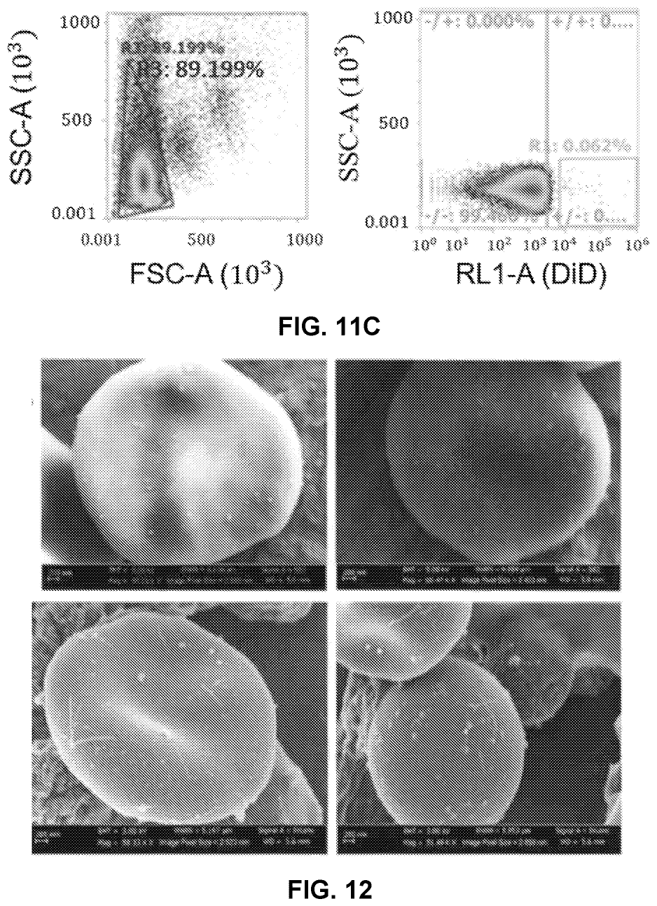

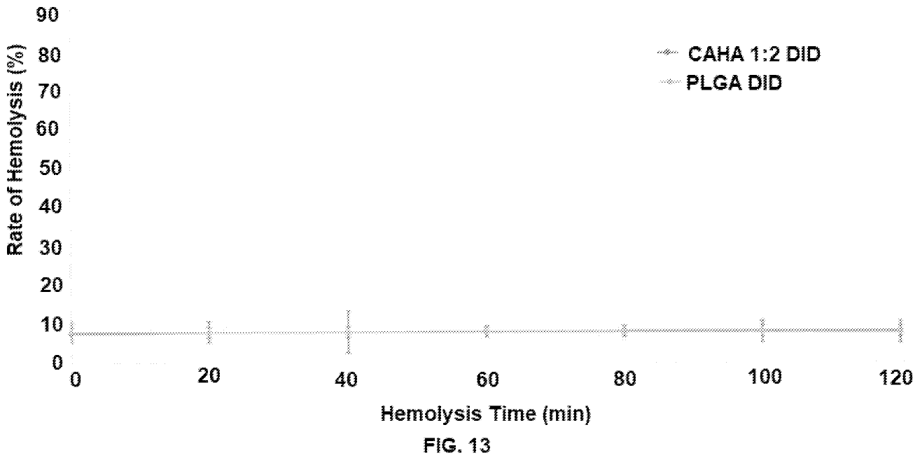

Biodistribution: After cardiac puncture exsanguination at the 6-hour endpoint to collect all remaining whole blood for flow cytometry detection (FIGS. 11A-11C) of circulating DiD-encapsulated NPs (RBC hitchhiking: FACS on isolated RBCs from whole blood performed according to conditions as previously described), whole blood-filtering organs were then collected and flash-frozen in triplicate (in dry ice and ethanol) and stored at −80° C. All organs were then massed and then homogenized at 10× scale (by mass) in 50% (v/v) Trichloroacetic Acid (TCA) in 1×PBS (pH 7.4) (i.e., 1000 mg in 10 mL of buffer). After all tissues were homogenized into a fine solution, homogenates were centrifuged at 4000×g at 4° C. for 10 minutes. Supernatant was then collected, and stored on ice while each solution was dropwise titrated to pH 8.0 with 5M NaOH and 1×TBS (range of [1:0.4]-[1:0.6] ratio of supernatant to NaOH, and range of 680 μL-3 mL added 1×TBS, when titrating against each sample). Once neutralized, each sample was vortexed, and then 200 μL was removed from the center of each vial, complemented by a 200 μL calibration curve for DiD fluorescence (0.5 mg/mL to 0 mg/mL), and fluorescence of 200 μL of 2% DiD NPs used for the injection study. Fluorescent readings from the wells were then normalized to the frozen mass of each tissue and volume of neutralized buffer solution, then compared to that of the DiD NPs to generate a percent of injected dose (% ID/tissue). Data is represented as average with standard error of mean (SEM) and statistical tests were performed using a two-tailed t-test for two samples at a time (p<0.05).

Abacavir Detection in the Brain: In parallel with DiD-NP injections, Sprague-Dawley rats injected with IL-PLGA NPs (either without cargo or with 60 μg/mL ABC, n=3) were sacrificed at 6 hours post-injection, and whole organs were processed, and frozen post-mortem as described above in Biodistribution. In order to assess to areas of biodistribution of abacavir within the brain, the following regions were sliced and homogenized in a 1:1 mixture of 1×PBS (pH 7.4) and Acetonitrile constituting a total homogenate volume of 500 μL per sample: striatum, midbrain, interbrain, cerebellum, hypothalamus, cerebral cortex, & hippocampus. To release NPs entrapped within the brain tissue, 500 μL RIPA lysis extraction buffer was first added to each homogenate [1:1 v/v] in a 1.5 mL Eppendorf centrifuge tube and brought to a final volume of 1 mL. All homogenates were then sonicated at 25° C. for 1 hour (with ice cubes added to prevent temperature elevation), followed by vortexing each sample at the highest setting for 10 minutes (in 2-minute intervals). Next, to assist in rupturing any extracted NPs, brain-region homogenates were stored at −80° C. for 2 minutes, vortexed at 2 minutes at 25° C., followed by centrifugation at 4° C. and 17,850 RPM for 15 minutes to fully break open and extract any remaining nanoparticle material and/or abacavir cargo from the tissue pellet. Lastly, each brain homogenate was shaken by inversion to resuspend the pellet and supernatant and filtered by 30 kDa centrifugation at 4500 RPM at 4° C. for 1 hour to extrude filtrate liquid smaller than 30 kDa through the tissue to the bottom of the tube. A range of 500-600 μL of liquid filtrate was recovered across all samples, which were all delivered into clean NMR tubes and topped 10% (v/v) with D₂O to be evaluated for presence of drug by ¹H NMR at 400 MHz with 90% H₂O/10% D₂O water suppression.

Microglial Extraction and FACS: To confirm microglial-specific uptake observed in the brain by confocal microscopy, rats injected with either saline (n=1) or IL-PLGA DID NPs (n=1) were evaluated for quantitative microglial uptake by fluorescence activated cell sorting (FACS) which was followed exactly according to a previously published protocol. Briefly, both control and IL-PLGA DID NP-treated rats were perfused with 1×HBSS (Thermo Fisher, #14175079) 6 hours post-injection, and extracted whole brains were submerged and frozen at −80° C. in 7 mL of phenol-red-free RPMI 1640 buffer (Gibco, cat. No. 11835, 500 mL). Whole brains were then homogenized in a 15 ml conical tube until a fine solution in the RPMI buffer, and then treated and layered with 3 mL 100% and 2 mL 70% Percoll (Sigma Aldrich, #17-0891-02) to isolate microglia via a density gradient post-centrifugation (30 minutes at 550×g and 18° C.). Isolated cells were then washed in 1×HBSS/10 mM HEPES (Gibco, #15630) via centrifugation for 7 minutes at 900×g and 4° C. and removed of myelin debris via Myelin Removal beads (Miltenyi Biotec, #130-096-733) and MACS separation system (Miltenyi Biotec, #130-042-108). Purified microglia were then labelled by CDIIb+ rat microbeads (cat #130-105-634) via MACS separation system, and then stained for FACS via FITC anti-rat CD11b/c Antibody (BioLegend, #201805). Three internal aliquots were run per sample (NaCl vs. IL-NP) at the lowest flow rate (12.5 μL/min) and 100,000 count-rate. Side scatter vs. forward scatter (SSC-A vs. FSC-A) was first used to identify scattering cells, which were gated and examined for singlet scattering (FSC-H vs. FSC-A) before identifying FITC-CDIIb+ microglia (BL1-A vs. FSC-A) and gating the CDIIb+ high population against far-red DiD fluorescence (BL1-A vs. RL1-A).

Confocal Fluorescence: Perfused and cryopreserved rat brains were sectioned using a microtome (Leica Biosystems, #CM3050S) at 20-micron thickness. The slides were washed twice with 1×PBS and permeabilized (with 0.1% Triton-X, 0.1% BSA in PBS) for 30 min at 25° C. The permeabilized slides were blocked (1% BSA, 1% normal goat serum in PBS) for an additional 30 min at 25° C. and followed by probing with primary rabbit-anti-IBal (1:200 dilution) or Mouse-GFP (1:250 dilution) for 90 min at 25° C. At the end of the incubation, the slides were washed 3x with PBS and followed by incubation with secondary antibodies (Texas Red Goat-anti Rabbit, 1:500 for IBa1 and Alexa 488 goat anti-mouse, 1:500 for GFP), 1 hr at 25° C. To stain cellular nuclei, Hoechst stain was used at 1:10000 dilution. All dilutions represented as (vol/vol). The slides were washed 3× with 1×PBS and mounted with antifade media and kept at 4° C. for further analysis with confocal microscopy.

### Example 2: Results and Discussion

### Nanoformulation & Characterization of IL-PLGA NPs for Brain Delivery of ARTs Via Intravenous (IV) Administration

We first prepared carboxylic acid-terminated poly(lactic-co-glycolic) acid (PLGA)-based nanoparticles (NPs) by nanoprecipitation and solvent evaporation of acetonitrile (ACN) as previously described, and as detailed in the Methods section. This produced bare, unloaded particles of 45.1±4.8 nm hydrodynamic diameter, −26.6±5.2 mV surface charge, and 0.17±0.06 polydispersity index (PDI) by Dynamic Light Scattering (DLS, n=4). The particles were then loaded with either equivalent amounts of abacavir (ABC) or far-red fluorescent dye 1,1′-dioctadecyl-3,3,3′,3′-tetramethylindodicarbocyanine, 4-chlorobenzenesulfonate (DiD) at approximately 2% by weight of the polymer (normalized to cargo molecular weight) into the organic phase. ABC-loaded NPs (n=7) had diameters of 76.4±13.5 nm, had a stable surface charge of −36.9±9.8 mV, and a monodisperse PDI of 0.09±0.04, while the DiD-loaded bare NPs (n=3) were 61.6±1.3 nm, −24.8±0.26 mV, and 0.11±0.015 PDI, indicating slightly improved loading abilities for ABC over DiD.

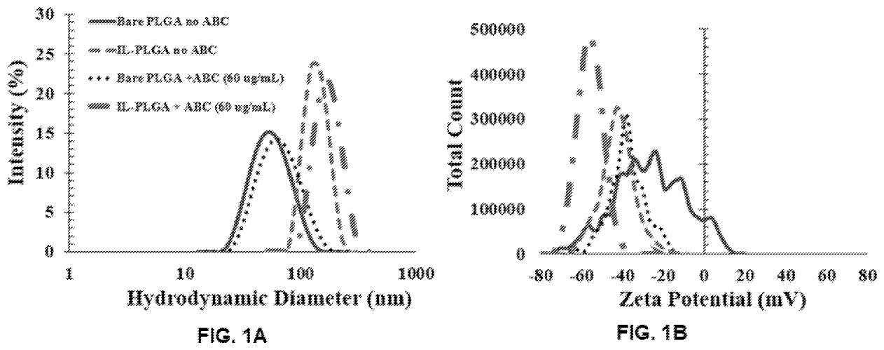

The loaded bare NPs were then coated with choline 2-hexenoate (CA2HA 1:2) IL (IL-PLGA NPs) by placing a single ~10 mg liquid drop in the center of the vial (10 mg neat IL/mg PLGA) and were stirred for 2 more hours. In each case, the previously bare NPs increased in size, and decreased in surface charge while maintaining a monodisperse PDI below 0.2. ABC-loaded IL-coated NPs were 191.5±23.6 nm, −54.8±6.5 mV, and had a PDI of 0.12±0.07 (n=5). FIGS. 1A-1D show the size (FIG. 1A) and surface charge (FIG. 1B) of the bare empty PLGA, ABC-loaded PLGA, IL-coated empty PLGA, and ABC-loaded IL-coated PLGA, as well as (FIG. 1C) bare and (FIG. 1D) IL-coated NP morphology by Scanning Electron Microscopy (SEM). Full DLS data is detailed in Table 1.

TABLE 1
Summarized DLS Data for Synthesized NPs

| | Size | Surface Charge | Polydispersity |
|---|---|---|---|
| Treatment | [nm] | [mV] | index [PDI] |
| PLGA (empty) (n = 4) | 45.1 ± 4.8 | −26.6 ± 5.2 | 0.17 ± 0.06 |
| IL-PLGA (empty)(n = 4) | 142.0 ± 5.6 | −43.9 ± 3.9 | 0.07 ± 0.03 |
| PLGA (dye) (n = 3) | 61.6 ± 1.3 | −24.8 ± 0.26 | 0.11 ± 0.015 |
| IL-PLGA (dye) (n = 4) | 164.6 ± 10.5 | −53.0 ± 12.9 | 0.18 ± 0.03 |
| PLGA (ABC) (n = 7) | 76.4 ± 13.5 | −36.9 ± 9.8 | 0.09 ± 0.04 |
| IL-PLGA (ABC) (n = 5) | 191.5 ± 23.6 | −54.8 ± 6.5 | 0.12 ± 0.07 |

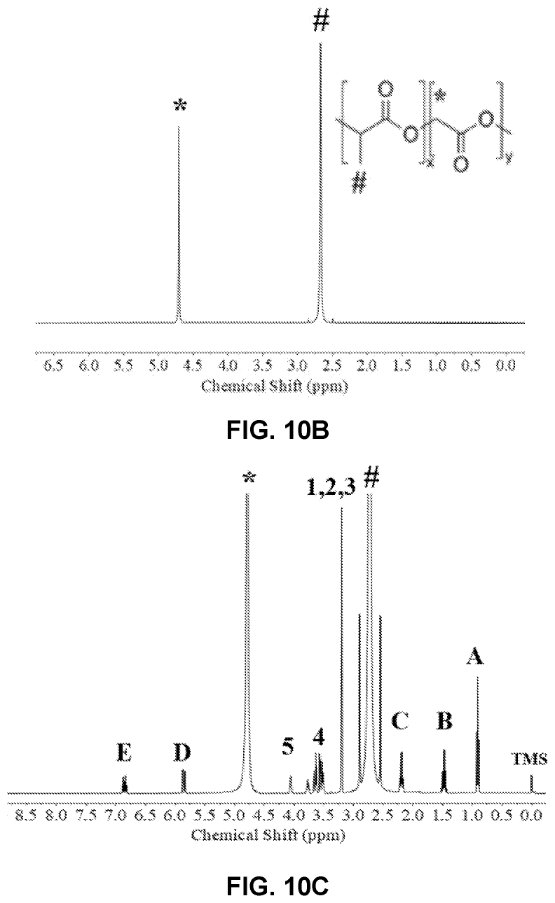

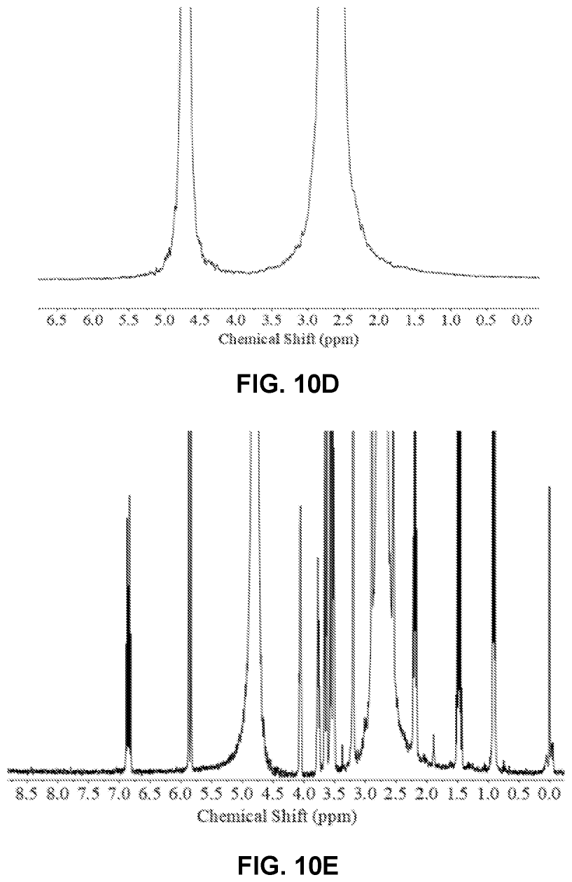

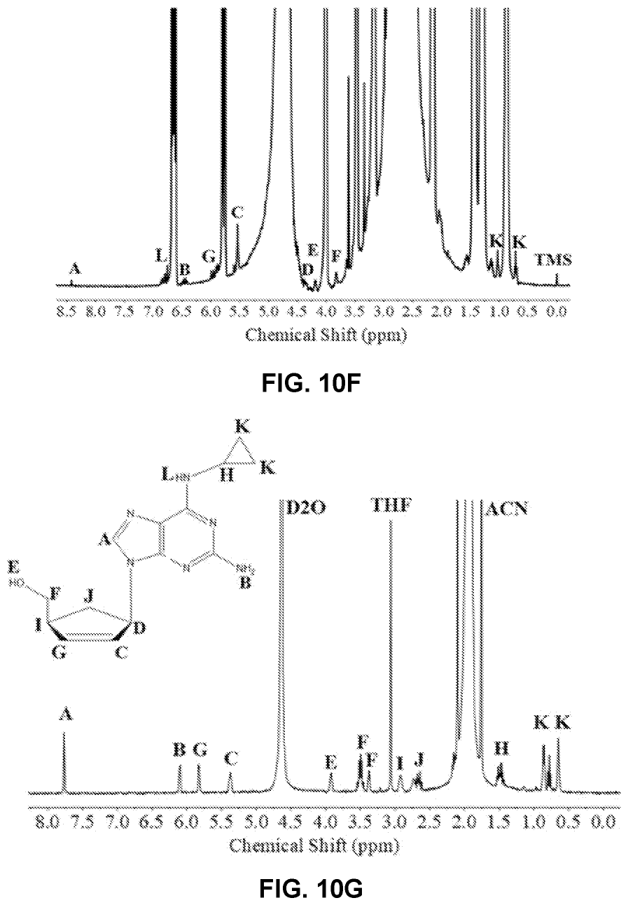

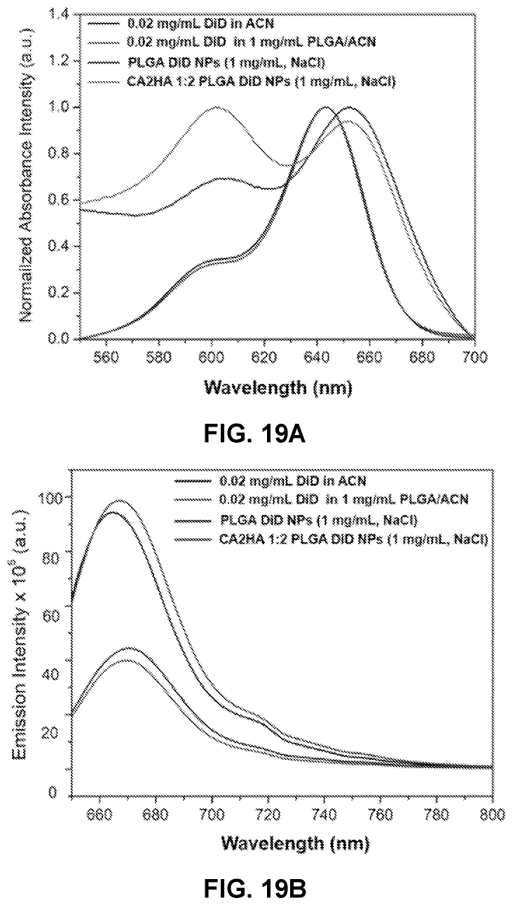

The encapsulation efficiency (EE) of DiD was determined by fluorescent plate reader (FIGS. 19A-19B) (DiD, 2% (v/v): 60.43±2.03% (n=3)), while the presence of ABC was measured by quantitative ¹H NMR spectroscopy (FIGS. 10A-10G) (ABC, 2% (v/v), estimated ~62.7 μg/mL from an added 74.1 μg/mL, or ~84.6% EE). The quantitative difference in encapsulation efficiency discovered between DiD and ABC is consistent with the DLS findings (FIGS. 1A-1D).

ART-Encapsulated IL-PLGA NPs Suppress HIV Viral Replication, Enhance Cellular Uptake of, and Are Biocompatible with, Human Peripheral Blood Mononuclear Cells (PBMCs)

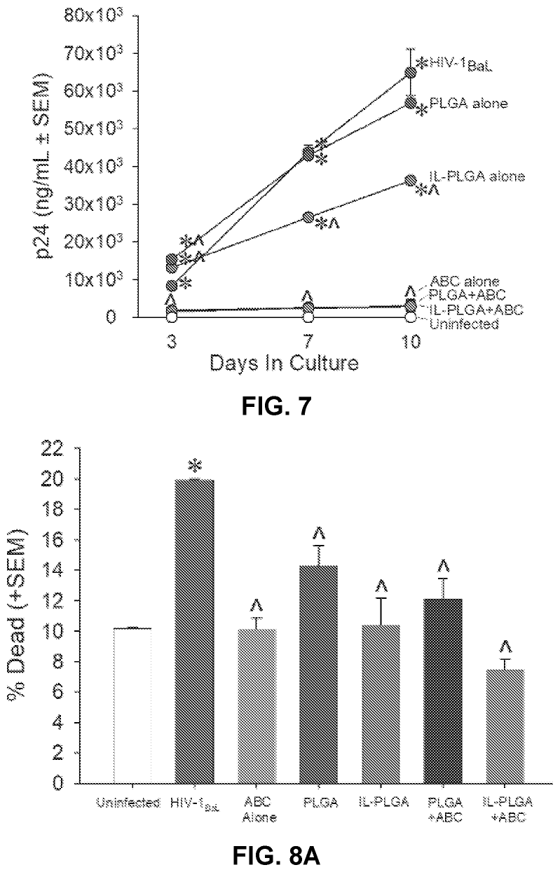

To assess the bioactivity of ART when encapsulated inside PLGA & IL-PLGA NPs, HIV-1 replication was assessed in human peripheral blood mononuclear cells (PBMCs) that were mock-infected or were infected with HIV-1BaL (1 ng/ml) for 10 days. PBMCs were treated with 1 mg/mL bare or IL-coated PLGA NPs that were either unloaded or loaded with abacavir (ABC, 60 μg/mL). ABC was also administered alone as a control. Concentration of the HIV-1 capsid protein (p24, ng/ml) was assessed on days 3-, 7-, and 10 post-infection by enzyme-linked immunosorbent assay (ELISA) (FIG. 7). As expected, viral replication was significantly greater in cells treated with HIV-1 alone, empty NPs, or empty IL-coated NPs compared to those that were mock-infected (p <0.0001-0.0009). Compared to HIV-infected cells, ABC significantly attenuated viral replication when administered alone and retained its bioactivity when encapsulated in NPs (p<0.0001). Intriguingly, IL-coated NPs significantly attenuated HIV-1 replication on their own (p=0.04); while ILs have previously demonstrated to exert virucidal effects, this has not been previously demonstrated with HIV-1.

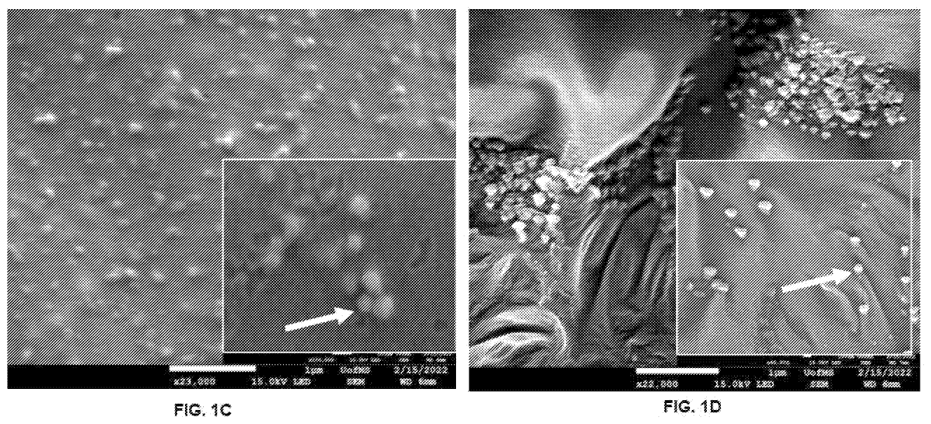

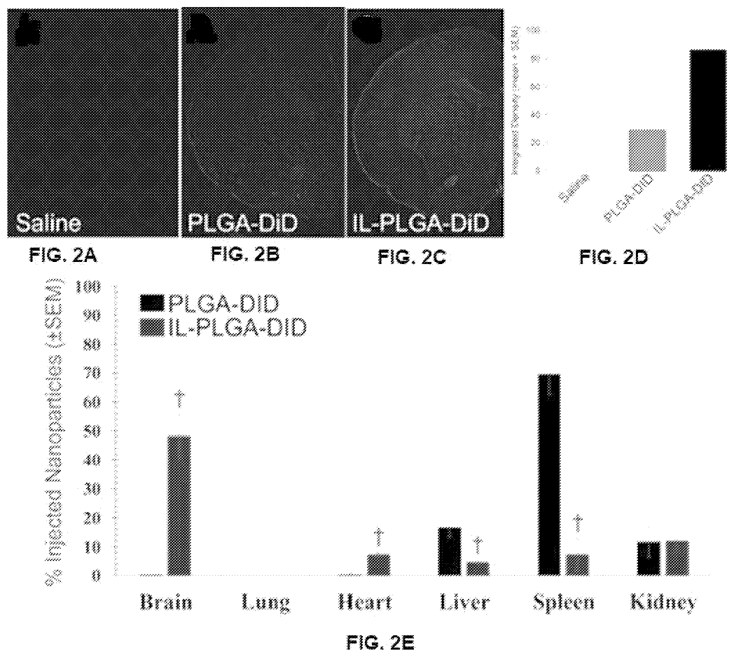

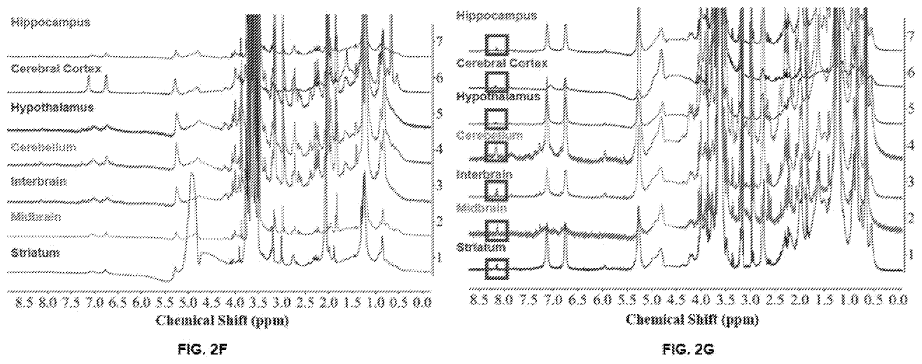

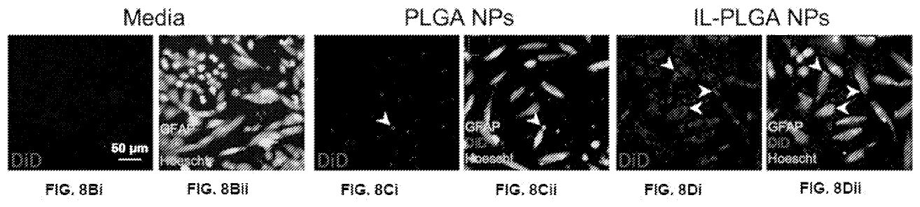

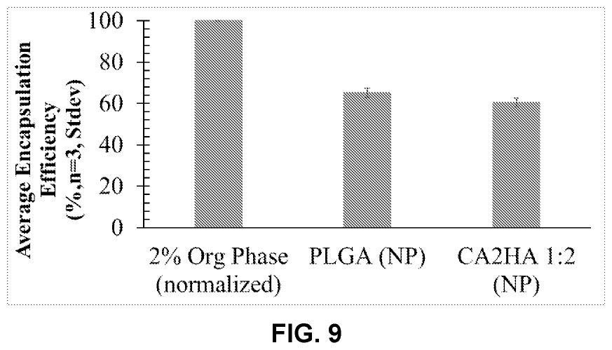

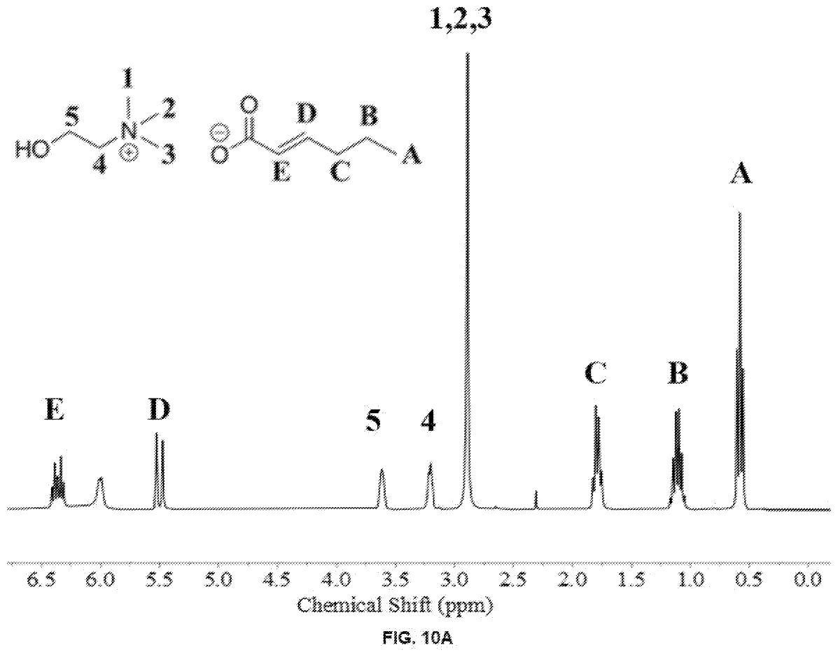

PBMC viability was also assessed at the 10-day timepoint via a trypan blue exclusion assay (FIG. 8A). As expected, HIV-1 significantly increased the proportion of dead cells (p=0.0004); any other treatment significantly attenuated this (p=0.0002-0.02). Additionally, when visualized by fluorescence microscopy, the uptake of DiD far-red fluorescent dye in co-cultured human astrocytes and human microglia cells (FIGS. 2Ci-2Eii) was dramatically enhanced when carried by IL-PLGA NPs (FIGS. 2Ei-2Eii) compared to bare PLGA NPs (FIGS. 2Di-2Dii) or media alone (FIGS. 2Ci-2Cii). This increased uptake could be partially explained by lipid extraction behavior engaged by the choline hexenoate coating, which has been shown before to increase uptake in RAW 264.7 macrophages, or the carboxylic anion interacting with the MCT-1 proteins present on the astrocytic cell surfaces.

### Carotid IV Injection Directs IL-PLGA NPs to the Brain and Results in Regional Abacavir (ABC) Brain Accumulation

To test the in vivo delivery efficacy, DiD-loaded bare and IL-coated PLGA NPs were intravenously (IV) injected into the carotid artery (500 μL) of healthy, 8-week-old, female, Sprague Dawley rats with in-dwelling carotid catheters (n=4/group). At 6 hrs, the rats were sacrificed and exsanguinated. Whole blood was collected by cardiac puncture and blood components were immediately analyzed by Fluorescence Activated Cell Sorting (FACS) (FIGS. 11A-11C). Blood-filtering organs were subsequently harvested (brain, lung, heart, liver, spleen, and kidneys) (n=3/group). From each treatment group, one animal underwent transcardial perfusion with phosphate buffer saline (1×PBS pH 7.4) followed by 4% PFA. One fixed brain from each group was used for epifluorescent imaging while the other three sets of organs were flash frozen and stored at −80° C. to perform biodistribution analysis. No physiologically-adverse effects of NPs were observed during live study or post-mortem.

As shown in FIGS. 2A-2E, notable differences in raw DiD signal were observed for IL-coated vs. uncoated DiD NPs in the brain via wide-field epifluorescence images (FIGS. 2A-2C). Compared to saline-infused rats (FIG. 2A), a faint DiD signal was detected in those infused with DiD-loaded NPs (FIG. 2B) compared to a much more intense signal for rats infused with IL-coated NPs (FIG. 2C; densitometric quantification in FIG. 2D). FIG. 2E shows the results of the quantitative biodistribution study (n=3/group, ±SEM). Bare PLGA NPs accumulated primarily in the spleen (69.6±6.9%), with a smaller amount in the liver (16.6±3.3%), kidneys (11.6±5.2%), and the least in the brain (0.1±0.1%). In contrast, the IL-coated NPs demonstrated the greatest accumulation in the brain (48.1±7.5%), with lesser concentration in the kidneys (12.1±3.2%), heart (7.3±0.8%), spleen (7.3±5.5%), and least in the liver (3.02±1.6%). However, there appeared to be no detectable accumulation in the lungs post-intracarotid injection. This finding contrasts with earlier work carried out via tail vein injection, suggesting that the target organ is critically dependent on the site of IV injection, which is consistent with prior RBC hitchhiking reports. The major innovation reported herein reveals the capacity for IL-NPs to hitchhike onto the RBCs post-injection, while earlier work required removal of the RBCs and NP attachment ex vivo. This is possible given that the IL coating imbues stealth properties onto the NP, allowing it to navigate the plasma and serum proteins to contact other blood components, even outperforming poly(ethylene) glycol.

Once biodistribution was determined with DiD, a new set of healthy, 8-week-old, female, Sprague Dawley rats with in-dwelling carotid catheters received IV infusions (under the same conditions) with either empty, or ABC-encapsulated, IL-coated PLGA NPs (n=3/group). Sites of regional distribution of abacavir cargo were evaluated within the brain (at the same 6-hour endpoint). Brain subregions (i.e., cerebral cortex, hippocampus, striatum, hypothalamus, midbrain, cerebellum, and interbrain) were grossly dissected, subsequently homogenized, and processed for assay via 1H-NMR spectroscopy to identify areas of selective ABC accumulation. As illustrated by both FIGS. 2F-2G, a broad range of proton peaks were found in the filtrate corresponding to those of the IL-PLGA NPs (such as the methyl or CH₂ group off the anion alkyl chain at 0.9 ppm and 1.5 ppm, or the protons off the trans-2-double bond between 6.5-7.5 ppm), albeit shifted due to the NP degradation process during extraction (when compared to intact IL-NPs in FIGS. 10C and 10E). While also slightly shifted due to the co-solvent composition during extraction, Abacavir's signature singlet proton peak is clearly distinguishable from the baseline at 8.1 ppm (FIGS. 10F-10G) in FIG. 10G, when compared to the empty cargo IL-NP delivery (FIG. 10F). Abacavir was observed to accumulate most greatly in the cerebellum, interbrain, striatum, and midbrain regions, with lesser (but considerable) delivery to the hippocampus, cerebral cortex, and hypothalamus regions (FIG. 10G). It seems likely that the intracarotid path of microvascular distribution contributed to this pattern of particulate accumulation, with IL-PLGA NPs shearing off from RBCs and subsequently crossing the BBB. Interestingly, as microglial populations are vastly diverse throughout these brain subregions, the potential for deep and comprehensive microglial targeting during HIV can be possible with such a distribution.

### IL-PLGA NPs Enter the Brain by Shearing Through Blood Vessels and Traffic to Microglia for Selective Uptake

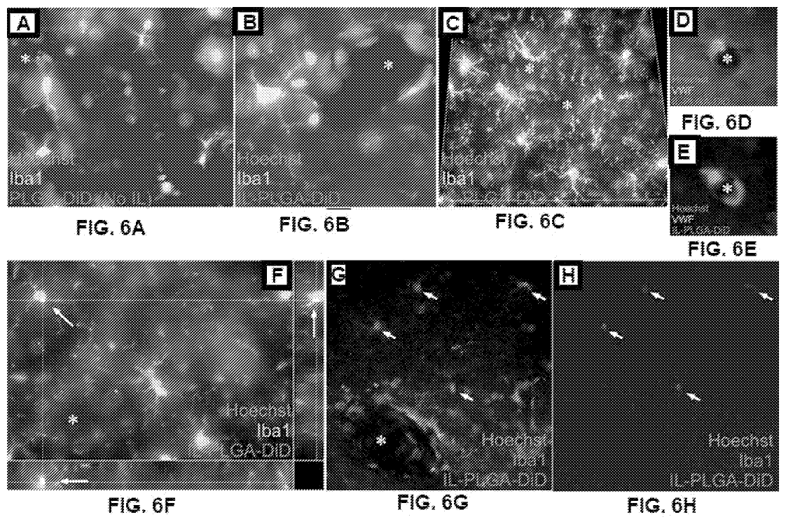

Bare PLGA NPs were only sparingly identified at the entrance of zoomed-in blood vessels in the brain via confocal microscopy of brain cross-sections (FIG. 6A). In contrast, magnitudes-greater IL-coated PLGA NPs were observed to enter into the brain through the vessel where they initially colocalized to endothelial cells (FIG. 6B), supporting the RBC hitchhiking-BBB shear theory. However, further throughout the caudate/putamen, IL-PLGA DiD NPs were able to actively migrate past endothelial cells after vessel entrance, where the majority was selectively & consistently uptaken into the soma of Iba-1+ microglia (FIGS. 6C and 6G-6H). However, to a lesser degree, IL-DID NPs also co-localized in von Willebrand factor-positive endothelial cells (FIGS. 6D-6E). To confirm microglial uptake vs. membrane-only adsorption, virtual cross-sections of Z-stacked images support the notion that DiD signal is located in the intracellular fraction of Iba-1+ cells (FIG. 6F).

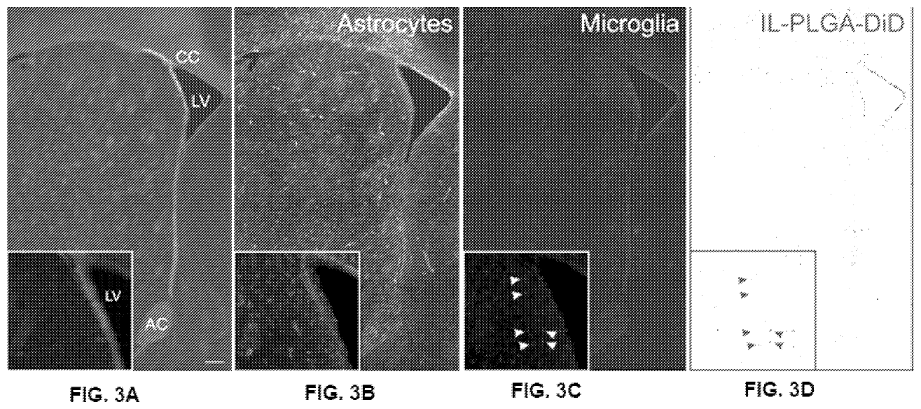

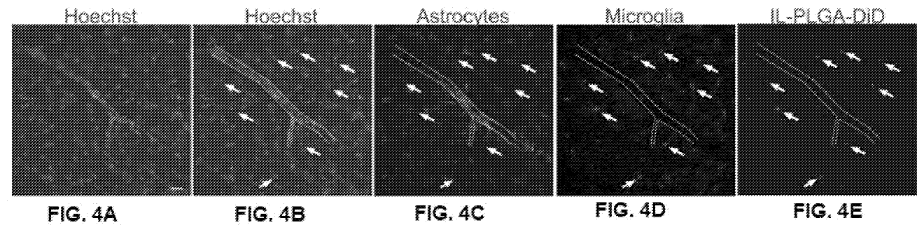

To both qualitatively and quantitatively confirm IL-PLGA NP selectivity for cells comprising the HIV reservoir, brain sections (40 μm) collected from rats used above were co-labeled for protein markers of astrocytes (GFAP; FIG. 3B) and microglia (Iba-1; FIG. 3C) with a Hoechst nuclear counterstain (FIG. 3A). Widefield images (10×) of the caudate/putamen within the dorsal striatum, a region of dense HIV viral load in the human brain, demonstrated an apparent colocalization of DiD signal (FIG. 3D) with Iba-1 (FIG. 3C) in the brain of a rat infused with IL-coated NPs. A cross-section of a blood vessel is seen (FIGS. 4A-4E; 20×) with the astrocytic component of the blood-brain barrier visualized (FIG. 4C). Surrounding microglia (FIG. 4D) are observed to colocalize with DiD signal (FIG. 4E).

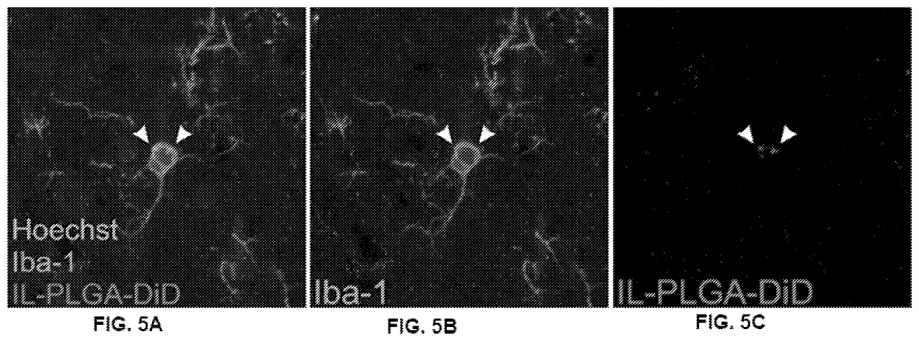

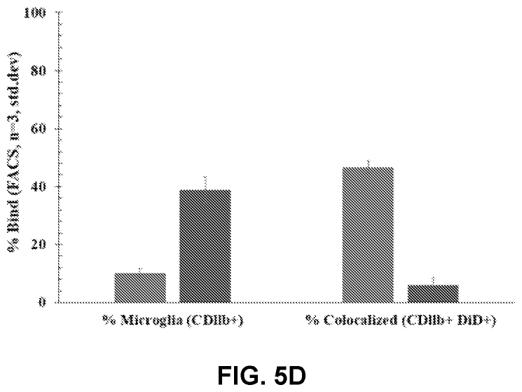

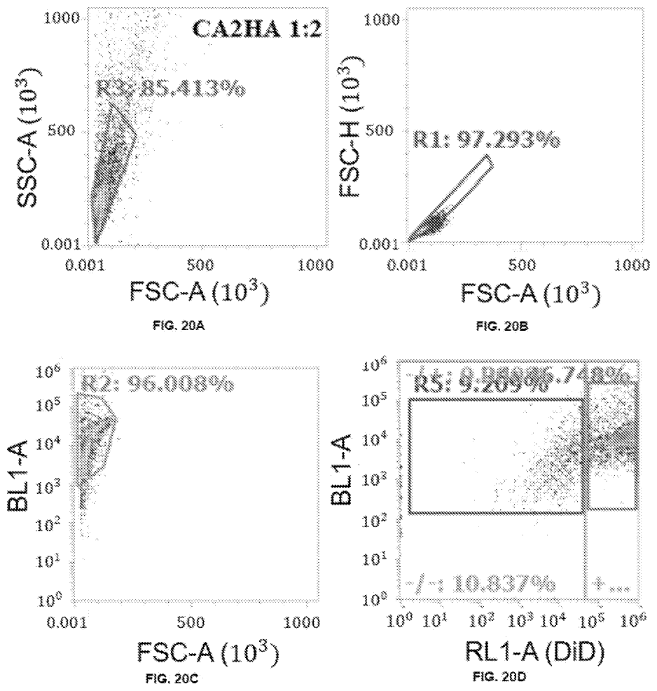

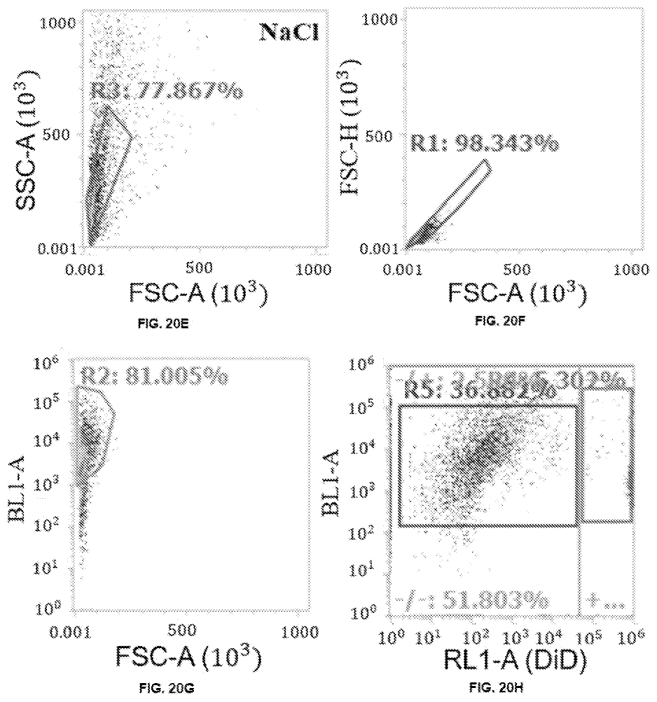

To ensure that spectral bleed-through from the near-infrared channel was not accounting for co-localization, Iba-1 was also assessed with a secondary antibody in the green wavelength of the electromagnetic spectrum and DiD signal was confirmed to co-localized with Iba-1+ cells (FIGS. 5A-5C). Interestingly, when microglia were extracted, isolated, and purified from whole Sprague-Dawley rat brains (at the 6-hour endpoint) treated with saline or IL-PLGA DID NPs (n=1/group with n=3 internal repetitions/group), fluorescence-activated cell sorting (FACS) indicated 46.5±2.3% of microglia were DiD+ (FIGS. 5D and 20A-20H), confirming selective microglial uptake identified by confocal imaging.

### CONCLUSIONS

We report a novel and highly-effective strategy for nanoparticle delivery to the brain through the development of bioinspired IL coatings that enable spontaneous hitchhiking onto red blood cells post-injection. In addition to tissue specificity, the IL coating also shows preferential uptake into microglia in vivo. The successful encapsulation of the ART, abacavir, has also been shown, and the in vitro assays herein indicate that the drug retains efficacy, the IL-NPs are non-toxic to PBMCs, and the IL coating significantly increases uptake of the NPs into cells. In all, bioinspired IL coatings are a promising new technology that could make delivery of a variety of therapeutics into the brain feasible and effective. Future studies will focus on determining efficacy in larger animal models, such as macaques, and in disease models, as well as safety and immune profiling.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Anselmo, A C et al. ACS Nano. 2013, 7, doi: 10.1021/nn404853z.
2. Asahchop, E L et al. Retrovirology. 2017, doi: 10.1186/s12977-017-0370-5.
3. Ash, M K et al. Vaccines. 2021, 9, doi: 10.3390/vaccines9080867.
4. Bertrand, L et al. J Neuroimmune Pharmacol. 2021, 16, doi: 10.1007/s11481-019-09858-x.
5. Brenner, J S et al. Nat Commun. 2018, 9, doi: 10.1038/s41467-018-05079-7.
6. Byrne, N et al. Phys. Chem. Chem. Phys. 2012, 14, DOI: 10.1039/c2cp41625e.
7. Chun, T W et al. Proc. Natl. Acad. Sci. 1997, 13193-13197.
8. Curreri, A M et al. Adv Sci. 2021, doi: 10.1002/advs.202004819.
9. Dash, P K et al. Nat Commun. 2019, 10, doi: 10.1038/s41467-019-10366-y.
10. De Scheerder, M A et al. Cell Host Microbe. 2019, 347-358.
11. Debernardi, R et al. J Neurosci Res. 2003, 73, doi: 10.1002/jnr.10660.
12. Fister, S et al. Front. Microbiol. 2017, 8, DOI: 10.3389/fmich.2017.01608.
13. Fister, S et al. Sep. Purif. Technol. 2015, 155, DOI: 10.1016/j.seppur.2015.03.035.
14. Fois, A F et al. Curr HIV/AIDS Rep. 2015, 299-303, doi: 10.1007/s11904-015-0257-9.
15. Garcia, J A et al. Curr Protoc Immunol. 2014, doi: 10.1002/0471142735.im1435s104.
16. Glassman, P M et al. Pharmaceutics. 2020, 12, doi: 10.3390/pharmaceutics12050440.
17. Hamadani, C M et al. Nanoscale. 2022, 14, doi: 10.1039/d2nr00538g.
18. Hamadani, C M et al. Nat Protoc. 2023, doi: 10.1038/s41596-023-00843-6.
19. Hamadani, C M et al. Sci Adv. 2020, doi: 10.1126/sciadv.abd7563.
20. Hanu, R et al. Am J Physiol Cell Physiol. 2000, 278, doi: 10.1152/ajpcell.2000.278.5.C921.
21. Kim, J T et al. Nat Commun. 2022, 13, doi: 10.1038/s41467-021-27647-0.
22. Ko, A et al. J Neuroimmune Pharmacol. 2019, 14, doi: 10.1007/s11481-018-9809-2.
23. Liu, R et al. Sci Transl Med. 2020, 12, doi: 10.1126/scitranslmed.aaz0802.
24. Lutgen, V et al. PLOS Pathog. 2020, doi: 10.1371/journal.ppat. 1008381.
25. Martinez-Picado, J et al. Curr Opin HIV AIDS. 2016, 11:417-423.
26. McMichael, A et al. Nature. 2001, 410, doi: 10.1038/35073658.
27. Moshikur, R M et al. Mol Pharm. 2021, 18, doi: 10.1021/acs.molpharmaceut. 1c00324.
28. Nath, A. J Neurovirol. 2015, 21, doi: 10.1007/s13365-014-0291-1.
29. Nurunnabi, M et al. Proc Natl Acad Sci USA. 2019, doi: 10.1073/pnas. 1914426116.
30. Osborne, O et al. Trends Neurosci. 2020, 695-708, doi: 10.1016/j.tins.2020.06.007.
31. Palanisamy, K et al. RSC Adv. 2022, 12, doi: 10.1039/d1ra08604a.
32. Patel, S H et al. Neurosci Lett. 2019, doi: 10.1016/j.neulet.2019.134475.
33. Rock, R B et al. Clin Microbiol Rev. 2004, 942-64, doi: 10.1128/CMR.17.4.942-964.2004.
34. Roosterman, D et al. AIMS Neurosci. 2020, 7, doi: 10.3934/Neuroscience.2020007.
35. Schmid, F et al. PLOS Comput Biol. 2019, 15, doi: 10.1371/journal.pcbi. 1007231.
36. Stratoulias, V et al. EMBO J. 2019, 38, doi: 10.15252/embj.2019101997.
37. Tanner, E E L et al. J Control Release. 2018, doi: 10.1016/j.jconrel.2018.07.029. Epub 2018 Jul. 17. PMID: 30026081.
38. Vaidya, A et al. J Control Release. 2020, doi: 10.1016/j.jconrel.2020.07.037.
39. Valdebenito, S et al. J Neurochem. 2021, 429-443, doi: 10.1111/jnc.15336.
40. Wallet, C et al. Front Cell Infect Microbiol. 2019, doi: 10.3389/fcimb.2019.00362.
41. Wang, Y L et al. Chem Rev. 2020, 13, doi: 10.1021/acs.chemrev.9b00693.
42. Williams, K C et al. Annu. Rev. Neurosci. 2002, 25:537-562.
43. Xiao, Q et al. Front Cell Infect Microbiol. 2019, doi: 10.3389/fcimb.2019.00069.
44. Xun, J et al. Retrovirology. 2021, 18, doi: 10.1186/s12977-021-00581-1.
29. Nurunnabi, M et al. Proc Natl Acad Sci USA. 2019, doi: 10.1073/pnas. 1914426116.
30. Osborne, O et al. Trends Neurosci. 2020, 695-708, doi: 10.1016/j.tins.2020.06.007.
31. Palanisamy, K et al. RSC Adv. 2022, 12, doi: 10.1039/d1ra08604a.
32. Patel, S H et al. Neurosci Lett. 2019, doi: 10.1016/j.neulet.2019.134475.
33. Rock, R B et al. Clin Microbiol Rev. 2004, 942-64, doi: 10.1128/CMR.17.4.942-964.2004.
34. Roosterman, D et al. AIMS Neurosci. 2020, 7, doi: 10.3934/Neuroscience.2020007.
35. Schmid, F et al. PLOS Comput Biol. 2019, 15, doi: 10.1371/journal.pcbi. 1007231.
36. Stratoulias, V et al. EMBO J. 2019, 38, doi: 10.15252/embj.2019101997.
37. Tanner, E E L et al. J Control Release. 2018, doi: 10.1016/j.jconrel.2018.07.029. Epub 2018 Jul. 17. PMID: 30026081.
38. Vaidya, A et al. J Control Release. 2020, doi: 10.1016/j.jconrel.2020.07.037.
39. Valdebenito, S et al. J Neurochem. 2021, 429-443, doi: 10.1111/jnc.15336.
40. Wallet, C et al. Front Cell Infect Microbiol. 2019, doi: 10.3389/fcimb.2019.00362.
41. Wang, Y L et al. Chem Rev. 2020, 13, doi: 10.1021/acs.chemrev.9b00693.
42. Williams, K C et al. Annu. Rev. Neurosci. 2002, 25:537-562.
43. Xiao, Q et al. Front Cell Infect Microbiol. 2019, doi: 10.3389/fcimb.2019.00069.
44. Xun, J et al. Retrovirology. 2021, 18, doi: 10.1186/s12977-021-00581-1.

1. A nanoparticle composition comprising a plurality of nanoparticles, each nanoparticle of the plurality comprising a core, wherein the core comprises a biocompatible copolymer, and each nanoparticle of the plurality further comprising an ionic liquid coating surrounding the core, wherein the nanoparticle composition is capable of crossing the blood brain barrier by selective binding of the ionic liquid coating to red blood cell membranes;
wherein the biocompatible copolymer comprises poly(lactic-co-glycolic acid), polycaprolactone-polyamidoamine (PCL-PAMAM), or any combination thereof; and
wherein the ionic liquid comprises choline and an anion derived from a saturated or unsaturated C2-C20 linear or branched fatty acid.

2. The nanoparticle composition of claim 1, wherein after crossing the blood brain barrier, the nanoparticle composition localizes in one or more cell types selected from red blood cells, monocyte-derived macrophages, microglia, perivascular macrophages, and astrocytes.

3. The nanoparticle composition of claim 1, wherein the fatty acid comprises a saturated fatty acid, a monounsaturated fatty acid, a polyunsaturated fatty acid, or any combination thereof.

4. The nanoparticle composition of claim 1, wherein the anion comprises butanoate, 2-butenoate, 3-butenoate, pentanoate, 2-pentenoate, 3-pentenoate, hexanoate, 2-hexenoate, 3-hexenoate, trans-2-methyl-2-pentenoate, heptanoate, 2-heptenoate, 3-heptenoate, octanoate, 2-octenoate, 3-octenoate, nonanoate, 2-nonenoate, 3-nonenoate, decanoate, 2-decenoate, 3-decenoate, undecanoate, 2-undecenoate, dodecanoate, fumarate, malonate, maleate, malate, acetoxyacetate, ethoxyacetate, 3-mercaptopropionate, or any combination thereof.

5. The nanoparticle composition of claim 4, wherein the anion comprises 2-hexenoate.

6. The nanoparticle composition of claim 1, wherein a molar ratio of the choline to the fatty acid is from about 1:1 to about 1:4.

7. (canceled)

8. The nanoparticle composition of claim 1, further comprising a pharmaceutical agent.

9. The nanoparticle composition of claim 8, wherein the pharmaceutical agent is present at from about 0.2 to about 10% (w/v) relative to a total volume of the nanoparticle composition.

10. (canceled)

11. The nanoparticle composition of claim 8, wherein the pharmaceutical agent comprises an anti-retroviral agent.

12. The nanoparticle composition of claim 11, wherein the antiretroviral agent comprises abacavir, emtricitabine, lamivudine, tenofovir disoproxil fumarate, zidovudine, doravirine, efavirenz, etravirine, nevirapine, rilpivirine, atazanavir, darunavir, fosamprenavir, ritonavir, tipranavir, enfuvirtide, maraviroc, cabotegravir, dolutegravir, raltegravir, fostemsavir, ibalizumab-uiyk, cobicistat, or any combination thereof.

13.-14. (canceled)

15. The nanoparticle composition of claim 1, wherein the nanoparticle composition is biocompatible.

16.-19. (canceled)

20. A method for treating a neurological disease or disorder in a subject, the method comprising administering the nanoparticle composition of claim 1 to the subject.

21.-23. (canceled)

24. The method of claim 20, wherein the composition is administered intravenously.

25. The method of claim 24, wherein a site of administration is intracarotid.

26. The method of claim 20, wherein from about 0.1 mg to about 5 mg of nanoparticles are administered per kg of subject body weight.

27. The method of claim 20, wherein the nanoparticle composition persists in the subject for about 48 hours or less.

28. (canceled)

29. The method of claim 20, wherein the neurological disease or disorder comprises neuroHIV, depression, addiction, migraine, schizophrenia, Alzheimer's disease, dementia, brain cancer, a lysosomal storage disease, traumatic brain injury, or any combination thereof.

30. The method of claim 20, wherein the nanoparticles accumulate in at least one cell type or organ associated with a neuroHIV reservoir.

31. The method of claim 30, wherein the organ comprises the brain, spleen, liver, bone marrow, thymus, lungs, lymph nodes, small intestine, colon, genital tract, or any combination thereof.

32. The method of claim 30, wherein the at least one cell type comprises microglia.
